(19) United States
(12) Patent Application Publication
(10) Pub. No.: US 2011/0151431 A1
(43) Pub. Date: Jun. 23, 2011

(54) DETECTION OF XENOTROPIC MURINE LEUKEMIA VIRUS

(75) Inventors: Judy A. Mikovits, Reno, NV (US); Vincent Lombardi, Reno, NV (US); Sandra Ruscetti, New Market, MD (US); Francis Ruscetti, New Market, MD (US); Robert Silverman, Beachwood, OH (US)

(73) Assignee: Whittemore Peterson Institute for Neuro-Immune Disease, Sparks, NV (US)

(21) Appl. No.: 12/818,893

(22) Filed: Jun. 18, 2010

Related U.S. Application Data
(63) Continuation-in-part of application No. 12/575,467, filed on Oct. 7, 2009.
(60) Provisional application No. 61/268,933, filed on Jun. 18, 2009.
(60) Provisional application No. 61/225,000, filed on Jul. 13, 2009.
(60) Provisional application No. 61/225,877, filed on Jul. 15, 2009.
(60) Provisional application No. 61/228,616, filed on Jul. 26, 2009.
(60) Provisional application No. 61/228,624, filed on Jul. 27, 2009.
(60) Provisional application No. 61/249,486, filed on Oct. 7, 2009.
(60) Provisional application No. 61/318,392, filed on Mar. 29, 2010.
(60) Provisional application No. 61/225,877, filed on Jul. 15, 2009.

(51) Int. Cl. C12Q 1/70

(52) U.S. Cl. 435/5

(57) ABSTRACT

Methods of detecting, diagnosing, monitoring or managing an XMRV-related neuroimmune disease such as chronic fatigue syndrome or XMRV-related lymphoma such as mantle cell lymphoma in a subject are disclosed. These methods comprise determining presence, absence or quantity an XMRV nucleic acid in a sample from a subject.

### CROSS-REFERENCE TO RELATED APPLICATIONS

This application is a Continuation-in-Part of U.S. Non-Provisional application Ser. No. 12/575,467, filed on Oct. 7, 2009; and the present application also claims priority to U.S. Provisional Application Ser. Nos. 61/268,933, filed on Jun. 18, 2009; 61/225,000, filed on Jul. 13, 2009; 61/225,877, filed on Jul. 15, 2009; 61/228,616, filed on Jul. 26, 2009; 61/228,624, filed on Jul. 27, 2009; 61/249,486, filed on Oct. 7, 2009; and 61/318,392, filed on Mar. 29, 2010; each of which is incorporated herein by reference in its entirety.

### STATEMENT REGARDING FEDERALLY SPONSORED RESEARCH OR DEVELOPMENT

This invention was made in part with Government support under U.S.P.H.S. Grant HHSN26120080001E and Grant NCI/NIH CA104943 awarded by the National Institutes of Health, and Grant W81XWH-07-1338 awarded by U.S. Department of Defense Prostate Cancer Research Program. The U.S. government has certain rights in the invention.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present invention generally relates to xenotrophic murine leukemia virus-related virus and detection thereof.

### BACKGROUND

Multiple Sclerosis (MS), Amyotrophic Lateral Sclerosis (ALS), Niemann-Pick Type C Disease, fibromyalgia, autism, chronic lyme disease, and Chronic Fatigue Syndrome (CFS) are examples of neurological diseases believed to involve malfunctions in the immune system.

Chronic fatigue syndrome (CFS) is a debilitating disease that affects more than one million people in the US alone. CFS is a disease characterized by severe and debilitating fatigue, sleep abnormalities, impaired memory and concentration, and musculoskeletal pain. In the Western world, the population prevalence is estimated to be of the order of 0.5%-2% (Papanicolaou et al. 2004. Neuroimmunomodulation 11(2):65-74; White. 2007. Popul Health Metr 5(1):6). CFS subjects are known to have a shortened life-span and are at risk for developing lymphoma. Currently, there is no diagnostic test and no treatment, except for the specific treatment of microbial infections in those cases in which microbial agents can be identified (Devanur and Kerr. 2006. J Clin Virol 37(3):139-150). Although the precise pathogenesis of CFS is unknown, a range of factors have been shown to contribute (Komaroff and Buchwald. 1998. Annu Rev Med 49:1-13; Devanur and Kerr. 2006. supra).

Several retroviruses such as the MuLVs, primate retroviruses, HIV and HTLV-1 are associated with cancer and neurological diseases (C. Power, Trends in Neurosci. 24, 162, 2001; Miller and Meucii 1999 TINS 22(10), 471-479; Power et al. 1994 Journal of Virology 68(7) 4463-4649). Investigation of the molecular mechanism of retroviral induced neurodegeneration in rodent models revealed vascular and inflammatory changes mediated by cytokines and chemokines and these changes were observed prior to any neurological pathology (X. Li, C., Hanson. J. Cmarik, S. Ruscetti J. Virol. 83, 4912, March, 2009, K. E. Peterson., B Chesebro. Curr. Opin. Microbiol. Immunol. 303, 67 2006). Neurological maladies and upregulation of inflammatory cytokines and chemokines are some of the most commonly reported observations associated with CFS. Retroviral involvement has long been suspected not only for CFS but also for other neurological diseases such as Multiple Sclerosis (MS) and Amyotropic Lateral Sclerosis (ALS) (E. DeFreitas et al., Proc Natl Acad Sci USA 88, 2922 (Apr. 1, 1991); A. Rolland et al., J Neuroimmunol 160, 195 (March, 2005); A. J. Steele et al., Neurology 64, 454 (Feb. 8, 2005)).

A lymphoma such as Mantle Cell Lymphoma (MCL) is a follicular lymphoma characterized by proliferation of atypical small lymphoid cells in wide mantles around benign germinal centers. (Weisenburger, D. D., et al., Blood 87: 4483-4494, 1996; Weisenburger, D. D., et al., Cancer 49: 1429-1438, 1982). MCL has been difficult to treat (Zelenetz, A. D., Annals of Oncology 17 (Supplement 4): iv12-iv14, 2006).

The gammaretrovirus Xenotropic Murine Leukemia Virus-Related Virus (XMRV) has recently been implicated in prostate cancers (Dong, B., et al., Proc. Nat'l. Acad. Sci. USA 104, 1865-1660, 2007; PCT patent application PCT/US2006/013167, published as PCT publication number WO2006110589 of Silverman et al.).

McCormick et al. recently explored the candidacy of XMRV in ALS; however, they did not find XMRV in the blood or cerebro-spinal fluid (CSF) of the 25 ALS patients where reverse transcriptase (RT) was detected (McCormick, A. L., et al., Neurology 70: 278, 2008).

Seroconversion is the development of detectable specific antibodies to microorganisms in the blood serum as a result of infection or immunization. Serology (the testing for antibodies) is used to determine antibody positivity. Prior to seroconversion, the blood test is seronegative for the antibody; after seroconversion, the blood test is seropositive for the antibody. Villinger et al. (AIDS Research and Human Retroviruses 2009, abstract OP-58, page 60), described seroconversion in Rhesus Macaques in response to XMRV infection using a Western Blot assay; specifically, an antibody response to env and gag proteins was reported in Rhesus Macaques. Based on these results, a double-antigen sandwich assay was developed to detect seroconversion events in both macaque and human. However, these findings have not been extended to detection of seroconversion antibodies against XMRV in humans.

### SUMMARY OF THE INVENTION

One aspect provides a method of detecting, diagnosing, monitoring or managing an Xenotropic Murine Leukemia Virus-Related Virus (XMRV)-related neuroimmune disease or an XMRV-related lymphoma in a subject by detecting presence, absence, or quantity of an XMRV polynucleic acid in a sample of the subject. In some embodiments, detecting presence, absence, or quantity of an XMRV polynucleic acid in a sample of the subject comprises contacting a sample of a subject and at least one nucleobase polymer under conditions sufficient for hybridization to occur between the at least one nucleobase polymer and an XMRV nucleic acid, or complement thereof, if present in the sample; and detecting presence, absence or quantity of a hybridization complex comprising the nucleobase polymer and an XMRV nucleic acid, or complement thereof; wherein the at least one nucleobase polymer comprises a sequence that hybridizes to a nucleic acid sequence comprising at least about 10 contiguous nucleotides of an XMRV nucleic acid, or complement thereof.

In some embodiments, the subject is a person having, suspected of having, or at risk for developing an XMRV-related neuroimmune disease or an XMRV-related lymphoma. In some embodiments, the subject exhibits signs and/or symptoms of a neuroimmune disease and/or a lymphoma.

In some embodiments, the neuroimmune disease is selected from the group consisting of Chronic Fatigue Syndrome (CFS), fibromyalgia, Multiple Sclerosis (MS), Parkinson's Disease, Amyotrophic Lateral Sclerosis (ALS), Niemann-Pick Type C Disease, autism spectrum disorder (ASD), and chronic lyme disease. In some embodiments, the lymphoma is selected from the group consisting of a XMRV-related Mantle Cell Lymphoma (MCL) or a Chronic Lymphocytic Leukemia lymphoma (CLL). In some configurations, the neuroimmune disease is selected from the group consisting of Chronic Fatigue Syndrome (CFS).

In some embodiments, the sample is selected from the group consisting of a blood sample, a serum sample, a plasma sample, a cerebrospinal fluid sample, and a solid tissue sample. In some embodiments, the sample comprises cells selected from the group consisting of fibroblasts, endothelial cells, peripheral blood mononuclear cells, and haematopoietic cells, or a combination thereof.

In some embodiments, the method includes selecting or modifying a treatment on the basis of detection of the presence, absence, or quantity of a XMRV polynucleic acid in a sample of the subject. In some embodiments, the method includes administering to the subject a therapeutically effective amount of an anti-viral compound if a hybridization complex is detected.

In some embodiments, the conditions sufficient for hybridization to occur consists of high stringency hybridization conditions. In some embodiments, the sequence that hybridizes to a nucleic acid sequence comprising at least 10 contiguous nucleotides of an XMRV nucleic acid, or a complement thereof, comprises the complement of a sequence having at least 80% sequence identity with a sequence comprised by an XMRV virus nucleic acid, or complement thereof.

In some embodiments, the sequence that hybridizes to a nucleic acid sequence comprising at least 10 contiguous nucleotides of an XMRV nucleic acid, or a complement thereof, comprises the complement of a sequence having at least 95% sequence identity with a sequence comprised by an XMRV virus nucleic acid, or complement thereof. In some embodiments, the sequence that hybridizes to a nucleic acid sequence comprising at least 10 contiguous nucleotides of an XMRV nucleic acid, or a complement thereof, comprises the complement of a sequence comprised by an XMRV virus nucleic acid, or complement thereof.

In some embodiments, the nucleobase polymer comprises DNA, RNA, or a nucleic acid analogue. In some embodiments, the nucleobase polymer further comprises a label. In some configurations, the label is selected from the group consisting of a radioisotope, a chromogen, a chromophore, a fluorophore, a fluorogen, an enzyme, a quantum dot and a resonance light scattering particle. In some embodiments, detecting presence, absence or quantity of the hybridization complex comprises detecting presence, absence or quantity of the label.

In some embodiments, detecting presence, absence or quantity of the hybridization complex comprises a hybridization assay selected from the group consisting of a Southern hybridization assay, a Northern hybridization assay, a dot-blot hybridization assay, a slot-blot hybridization assay, a Polymerase Chain Reaction (PCR) assay and a flow cytometry assay. In some configurations, the PCR assay is a quantitative real time polymerase chain reaction assay. In some configurations, the PCR assay comprises one or more primers selected from the group consisting of SEQ ID NOS: 5-20.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a series of gel images showing XMRV sequences in peripheral blood mononuclear cell (PBMC) DNA from CFS subjects. Single round PCR for gag, env and gapdh sequences in PBMC of (FIG. 1A) CFS subjects and (FIG. 1B) normal health control individuals. The positions of the amplicons are indicated and DNA markers (ladder) are shown. Further details regarding methodology are provided in Example 7.

FIG. 2 is a series of line plots and gel and micrograph images showing XMRV protein expression in CFS subject PBMC. (FIG. 2A) Subject PBMC were activated on PHA and IL-2 for 7 days using a monoclonal antibody to SFFV p30. (FIG. 2B) Activated subject PBMC were assayed with anti goat antisera against purified gp70, P30, p10 from Rauscher MuLV; Pre immune normal goat sera was used as a control. (FIG. 2C) Lysates prepared from PBMC activated with PHA and IL-2 from subjects WPI-1125, WPI-1150, WPI-1199, WPI-1220 and WPI-1221 (lanes 1-5, respectively) were analyzed by Western blot with rat anti-SFFV env monoclonal antibody (top panel), goat anti-NZB xenotropic MuLV (middle panel) or goat anti-R-MuLV p30 (bottom panel). Lane 6: lysates from SFFV-infected HCD-57 cells; lane 7: lysates from F-MuLV-infected NIH3T3 cells. (FIG. 2D) Lysates from normal PBMCs (lanes 1, 2, 4, 5 and 7) or from subjects WPI-1235 (lane 3) and WPI-1236 (lane 6) were activated for 7 days with PHA and IL-2 and then analyzed by Western blot using rat anti-SFFV env monoclonal antibody (top panel) or goat anti-R-MuLV p30 (bottom panel). Lane 8: SFFV-infected mouse HCD-57 cells. Molecular weight markers are shown on the left. (FIG. 2E) Left: CD4+ T cells were purified and activated and XMRV p30 was detected using anti p30 monoclonal. Right: CD19+ B cells were purified and activated as described in the methods; XMRV p30 expression detected after 14 Days using a monoclonal ab to SFFV p30. (FIG. 2F) From WPI-1105. Thin-sectioned analysis of virus particle showing electron dense core surrounded by an envelope. Scale bar for the micrograph is 100 nm. (FIG. 2G) A B-cell line from CFS subject markedly positive for XMRV gp70 detected by using rat anti SFFV gp55 with the normal hybridoma used as a control. (FIG. 2H) Lysates from T or B cells grown for 42 days on IL-2 or CD40L, respectively, were prepared and analyzed by Western blot using rat anti-SFFV env monoclonal antibody (top panel) or goat anti-NZB xenotropic MuLV (bottom panel). Lane 1: WPI-1118 B cells; lane 2: WPI-1104 T cells; lane 3: WPI-1106 T cells. Lane 4: normal T cells; lane 5: mouse HCD-57 cells; lane 6: SFFV-infected mouse HCD-57 cells; lane 7: F-MuLV-infected NIH3T3 cells. Molecular weight markers are shown on the left. Further details regarding methodology are provided in Example 8.

FIG. 3 is a series of Igel images, line plots, and micrograph images showing infectious and transmissable XMRV in CFS subject PBMCs. (FIG. 3A) Lanes 1, 3, 5: PBMC lysates from subjects WPI-1104, WPI-1150 and WPI-1221 transmitted to LNCaP; lanes 2 and 4: normal PBMCs; lane 6: uninfected LNCaP; lane 7: SFFV-infected mouse HCD-57 cells. Viral protein expression was detected by Western blotting using a rat anti-SFFV Env monoclonal antibody (top panel) or goat anti-NZB xenotropic MuLV (bottom panel). Molecular weight markers are shown on the left. (FIG. 3B) The indicated T-cell culture form CFS subject were co-cultured with LNCaP. XMRV p30 expression was detected by anti-SFFV p30 monoclonal antibody. (FIG. 3C) Thin-sectioned analysis of virus particle showing electron dense core surrounded by an envelope. Scale bar is 100 nm. Further details regarding methodology are provided in Example 9.

FIG. 4 is a series of gel images and line plots showing infectious XMRV in CFS subject plasma. (FIG. 4A) Plasma from subjects WPI-1178, WPI-1150, WPI-1118, WPI-1141, WPI-1197 and WPI-1206 (lanes 1-6, respectively) were co-cultured with LNCaP cells and lysates prepared after six passages. Viral protein expression was detected by Western blotting with rat anti-SFFV Env monoclonal antibody (top panel) or goat anti-R-MuLV p30 (bottom panel). Lane 7: uninfected LNCaP; lane 8: SFFV-infected mouse HCD-57 cells. Molecular weight markers are shown on the left. (FIG. 4B) Cell free transmission of XMRV to SupT1 cell line was demonstrated using Transwell (Costar) co-culture system. Lane 1: MW marker. Lane 2: SupT1 co-cultured with Raji. Lane 3: SupT1 co-cultured with subject WPI-1199. Lane 4: SupT1 co-cultured with WPI-1220. Lane 5: SupT1 co-cultured with WPI-1141. Lane 6: SupT1 co-cultured with WPI-1199. Lane 7: SupT1 co-cultured with WPI-1169. Lane 7: Raji alone. Lane 8: No template control (NTC). Lane 9: SupT1 co-cultured with WPI-1199. (FIG. 4C) Primary and secondary cell free transmission of XMRV from subjects' T cells to normal T cells. Normal T cells were infected with supernatants from T cells from subject WPI-1150 (lane 1); subject WPI-1118 (lane 4); subject WPI-1220 (lane 5), and subject WPI-1221 (lane 6). Lanes 7 and 8 are secondary transmission to normal T cells of supernatants from T cells infected with supernatants from subject WPI-1220 and WPI-1221, respectively. Lanes 2 and 3: uninfected T cells; Lane 9: SFFV-infected mouse HCD-57 cells. Viral protein expression was detected by Western blotting using a rat anti-SFFV env monoclonal antibody. Molecular weight markers are shown on the left. (FIG. 4D) Plasma from the indicated CFS subject was co-cultured with human foreskin fibroblasts (HFF). At the second passage, XMRV p30 was detected using a rat anti-SFFV p30 monoclonal antibody. Further details regarding methodology are provided in Example 10.

FIG. 5 illustrates sequence of gag from two subjects, amplified by nested PCR. Further details regarding methodology are provided in Example 7.

FIG. 6 illustrates partial sequences from CFS XMRV strains 1130, 1138 and 1169 in comparison to XMRV strains VP62, VP35 and VP42 derived from prostate cancers. Shadowed text indicates position difference. Further details regarding methodology are provided in Example 7.

FIG. 7 illustrates detection of cloned XMRV using a rat monoclonal antibody to SFFV gp55 Env. Lysates were prepared from XMRV infected Raji (lane 1), XMRV infected LNCaP (lane 2) or XMRV infected SupT1 (lane 3). Lane 4: mouse HCD-57 cells; lane 5: SFFV infected H D57 cells. Western blot analysis was carried out using rat anti-SFFV Env monoclonal antibody 7C10. Molecular weight markers are shown on the left. Further details regarding methodology are provided in Example 8.

FIG. 8 illustrates detection of cloned XMRV using goat antiserum to mouse NZB xenotropic MuLV. Lysates were prepared from XMRV-infected Raji (lane 1), XMRV infected LNCaP (lane 2) or XMRV infected Sup-T-1 (lane 3). Lane 4: SFFV infected mouse HD57 cells. Uninfected Raji, LNCaP and SupT1 are shown in lanes 5-7, respectively. Western blot analysis was carried out using goat anti-NZB xenotropic MuLV. Molecular weight markers are shown on the left. Further details regarding methodology are provided in Example 8.

FIG. 9 is a scatter plot showing presence of antibodies to SFFV-env in CFS subjects' plasma as the difference in mean fluorescence intensity (MFI) between CFS and control plasma direct binding to BaF3ER-SFFV Env cells versus BaF3ER (control) cells. BaF3ER-SFFV Env (MFI) is shown as a function of human plasma dilution (1:10) for subjects 1-9 (squares) and controls 1-7 (triangles). Further details regarding methodology are provided in Examples 12 and 14.

FIG. 10 is a series of graphs depicting CFS plasma antibody recognition of cell surface SFFV Env expressed in cell line BaF-3. FIG. 10A shows normal plasma. FIG. 10B shows WPI-1104 plasma. FIG. 10C shows α-SFFV Env. FIG. 10D shows α-SFFV Env, WPI-1104 plasma. Further details regarding methodology are provided in Example 13.

FIG. 11 is a series of graphs showing antibody reactivity in CFS plasma to SFFV-Env expressed in BAF3ER cells. FIG. 11A shows antibody reactivity to BaF3ER for normal plasma and WPI-1104 plasma; and control and α-SFFV Env. FIG. 11B shows antibody reactivity to BaF3ER-SFFV Env for normal plasma and WPI-1104 plasma; and control and α-SFFV Env. FIG. 11C shows antibody reactivity to SFFV Env in the presence of human plasma for control, α-SFFV Env, WPI-1104 plasma, and 1:10+α-SFFV Env; and control, α-SFFV Env, WPI-1104 plasma, and 1:100+α-SFFV Env. Further details regarding methodology are provided in Examples 12 and 14.

FIG. 12 is a series of line plots showing intracellular staining for XMRV Env using the SFFV env moAb (darker line, i.e., line shifted right at no AZT day 3) or isotype control (lighter line) for separated PBMC unactivated at time 0 or PHA/IL-2 activated in the presence and absence of 50 nM AZT for three days. Further details regarding methodology are provided in Examples 14 and 20.

FIG. 13 is diagram depicting the phylogenetic relationship among endogenous (non-ecotropic) MLV sequences, XMRV sequences, and sequences from CFS subjects 1104, 1106, and 1178. Further details regarding methodology are provided in Example 21.

### DETAILED DESCRIPTION OF THE INVENTION

The present disclosure is based, at least in part, on the observation that Xenotropic Murine Leukemia Virus-Related Virus (XMRV), a human gammaretrovirus, nucleic acids were detected in the peripheral blood mononuclear cells (PBMC) of 68 of 101 (67%) of subjects diagnosed with CFS whereas only 8 of 218 (3.7%) of regional, healthy controls tested positive for XMRV DNA. Furthermore, as shown herein, infectious virus was transmitted from activated primary PBMC as well from as purified B and T cell cultures and plasma derived from subjects diagnosed with CFS. Methods of transmitting XMRV comprise establishing a secondary infection in uninfected primary lymphocytes and indicator cell lines. Phylogenetic analysis supported that XMRV is a human gammaretrovirus. Full length sequencing revealed 99% sequence identity with previously isolated strains of XMRV. These results are supported by the transmission electron microscopy observation of type C retrovirus particles in subject PBMCs. Taken together, these data demonstrate direct isolation of infectious XMRV from humans. Furthermore, these data support a role for XMRV infection in the pathogenesis of a neuroimmune disease.

Accordingly, disclosed herein are methods of detecting, diagnosing, monitoring or managing a neuroimmune disease such as Multiple Sclerosis (MS), Amyotrophic Lateral Sclerosis (ALS), Niemann-Pick Type C Disease, Chronic Fatigue Syndrome (CFS), fibromyalgia or autism spectrum disorder (ASD), or chronic lyme disease; or a lymphoma, such as Mantle Cell Lymphoma (MCL) or Chronic Lymphocytic Leukemia lymphoma (CLL) in a subject. In particular, disclosed are methods of detecting, diagnosing, monitoring or managing a neuroimmune disease or a lymphoma in a subject who is infected with, is suspected of being infected with, or is at risk of infection with XMRV. Also described are methods of screening for anti-retroviral agents effective against XMRV or for management or treatment of an XMRV-related disease or disorder. Anti-retroviral compositions and their use in treatment of an XMRV-related disease or disorder are described. Cells, cell lines, and cell cultures that can be used in the various methods herein are also described. Vaccines, methods of preparing vaccines, and methods of conferring immunity are likewise described. Further details regarding these and other aspects and features follow.

Detection

One aspect provides a method for detecting XMRV in a subject or a sample from a subject. The method can be in conjunction with diagnosing, monitoring, or managing an XMRV-related disease or disorder, such, without limitation, a neuroimmune disease or an XMRV-related lymphoma. Detection of XMRV in a sample can be indicative, or confirmatory, of a diagnosis of an XMRV-related disease or disorder, such as an XMRV-related neuroimmune disease or an XMRV-related lymphoma. Monitoring can include detection or repeated detection of XMRV from a sample or samples of a subject in which XMRV has already been detected. Managing can include therapeutic intervention based upon the presence or absence of XMRV in a subject, as discussed further herein.

The neuroimmune disease can be any neuroimmune disease known to skilled artisans, such as, without limitation, Chronic Fatigue Syndrome (CFS), Multiple Sclerosis (MS) including atypical MS, Parkinson's Disease, Amyotrophic Lateral Sclerosis (ALS), Niemann-Pick Type C Disease, fibromyalgia, autism spectrum disorder (ASD), or chronic lyme disease. Examples of an XMRV-related neuroimmune disease include, but are not limited to Chronic Fatigue Syndrome (CFS), fibromyalgia, Multiple Sclerosis (MS), Parkinson's Disease, Amyotrophic Lateral Sclerosis (ALS), Niemann-Pick Type C Disease, autism spectrum disorder (ASD), and chronic lyme disease. For example, CFS can be detected, diagnosed, monitored, or managed by detecting XMRV in a subject or a sample from a subject. As another example, MS, such as Atypical Multiple Sclerosis (Batianello, S., et al., Neurol. Sci. Suppl. 4, S356-S360, 2004; Phadke, J. G. et al., J. Neurology, Neurosurgery and Psychiatry 46, 414-420, 1983), can be detected, diagnosed, monitored, or managed by detecting XMRV in a subject or a sample from a subject. Atypical MS can be an MS in which no demyelination is detected by standard procedures such as MRI scanning. An XMRV-related disease can be a neural disease, such as, without limitation, an XMRV-related neural disease that is not generally recognized as a neuroimmune disease, such as, for example, XMRV-related bipolar disorder or a neurodegenerative disease such as XMRV-related Alzheimer's disease or XMRV-related Parkinson's disease.

Examples of an XMRV-related lymphoma include, but are not limited to an XMRV-related Mantle Cell Lymphoma (MCL) and a Chronic Lymphocytic Leukemia lymphoma (CLL).

On the basis of the detection of the presence, absence or quantity of XMRV in a subject, a treatment can be selected, monitored, or modified. Detection of the presence, absence or quantity of XMRV in a subject can comprise detection of XMRV in a sample from the subject.

Sample and Subject

Detection methods described herein are generally performed on a subject or on a sample from a subject. A sample can contain or be suspected of containing XMRV. A sample can be a biological sample from a subject.

The subject can be a subject having, diagnosed with, suspected of having, or at risk for developing a neuroimmune disease or a lymphoma, such as an XMRV-related neuroimmune disease or an XMRV-related lymphoma. For example, a subject can be tested for the presence of an XMRV where the subject exhibits signs or symptoms of a neuroimmune disease or a lymphoma. As another example, a subject can have been diagnosed with a neuroimmune disease or lymphoma, or diagnosed with an XMRV-related neuroimmune disease or XMRV-related lymphoma. A subject can be considered at risk of developing a neuroimmune disease or lymphoma, such as, without limitation, an individual with a familial history of a neuroimmune disease or lymphoma, or an individual residing in a region comprising a cluster of individuals with a neuroimmune disease or lymphoma.

A determination of the need for detecting, diagnosing, monitoring, or managing an XMRV-related neuroimmune disease or an XMRV-related lymphoma will typically be assessed by a history and physical exam consistent with the disease or condition at issue. Such assessment is within the skill of the art. The subject can be an animal subject, preferably a mammal, more preferably horses, cows, dogs, cats, sheep, pigs, mice, rats, monkeys, guinea pigs, and chickens, and most preferably a human.

For example, a subject can be one which fulfills the 1994 CDC Fukuda Criteria for CFS (Fukuda et al., Ann Intern Med 1994; 121: 953-9); the 2003 Canadian Consensus Criteria (CCC) for ME/CFS (Carruthers et al, J Chronic Fatigue Syndrome 2003; 11:1-12; Jason et al., J Chronic Fatigue S 2004; 12:37-52), or both the Fukuda and CCC criteria. The CCC requires post-exertional malaise, which many clinicians believe is the sine qua non of ME/CFS. In contrast, the Fukuda and 1991 Oxford Criteria do not require exercise intolerance for a diagnosis of ME/CFS. The CCC further requires that subjects exhibit post-exertional fatigue, unrefreshing sleep, neurological/cognitive manifestations and pain, rather than these being optional symptoms.

As another example, the subject can be an animal, such as a laboratory animal that can serve as a model system for investigating a neuroimmune disease or lymphoma (see e.g., Chen, R. et al., Neurochemical Research 33: 1759-1767, 2008; Kumar, A., et al., Fundam. Clin. Pharmacol. Epub ahead of print, Jan. 10, 2009; Gupta, A., et al., Immunobiology 214: 33-39, 2009; Singh, A., et al., Indian J. Exp. Biol. 40: 1240-1244, 2002; Ford, R. J., et al. Blood 109: 4899-4906, 2007; Smith, M. R., et al., Leukemia 20: 891-893, 2006; Bryant, J., et al., Lab. Invest. 80: 557-573, 2000; M'kacher, R., et al., Cancer Genet Cytogenet. 143: 32-38, 2003).

A sample can be a blood sample, a serum sample, a plasma sample, a cerebrospinal fluid sample, or a solid tissue sample. For example, the sample can be a blood sample, such as a peripheral blood sample. As another example, a sample can be a solid tissue sample, such as a prostate tissue sample.

A sample can include cells of a subject. For example, a sample can include cells such as fibroblasts, endothelial cells, peripheral blood mononuclear cells, haematopoietic cells, or a combination thereof.

Probe to Detect Retroviral Antigen in a Sample

Detecting, diagnosing, monitoring or managing an XMRV-related neuroimmune disease or XMRV-related lymphoma in a subject can be based upon a probe that binds at least one gammaretrovirus antigen, such as an XMRV antigen.

In a probe-based method, a sample can be obtained from a subject. The sample or subject can be as discussed above. In some configurations, a reaction mixture is formed with a sample from a subject, or antigens thereof, and at least one probe that binds at least one gammaretrovirus antigen, such as an XMRV antigen. The reaction mixture can be exposed to conditions sufficient for formation of a complex between probe and antigen, if present. The presence, absence or quantity of a complex between the (supplied) probe and (sample) gammaretrovirus antigen, such as an XMRV antigen, can then be detected.

A probe that binds gammaretrovirus antigen, such as an XMRV antigen, in a sample can be, for example, an antibody, an antigen-binding fragment of an antibody, an aptamer (Jayasena, S. D., et al., Clinical Chemistry 45: 1628-1650, 1999), a kinase, an avimer (Silverman, J., et al., Nature Biotechnology 23: 1556-1561, 2005) or combinations thereof that bind XMRV, an XMRV antigen, or an antigen of a taxonomically related gammaretrovirus. For example, a probe that binds an XMRV antigen can be an antibody, such as a polyclonal antibody or a monoclonal antibody, or an antigen-binding fragment thereof, such as an Fab fragment. As another example, a probe that binds a gammaretrovirus antigen can be a polyclonal antibody against a virus that is taxonomically related to XMRV (e.g., Murine Leukemia Virus such as a Xenotropic Murine Leukemia Virus, i.e., Xenotropic MuLV). An exemplary polyclonal antibody can be against an NZB Xenotropic MuLV (O'Neill, R. R., et al., J. Virol. 53: 100-106, 1985). As another example, a probe that binds an XMRV antigen can be an anti gp 55 Env antibody. An exemplary monoclonal antibody that binds an XMRV antigen includes a monclonal antibody against p30 gag. An exemplary monoclonal antibody that binds an XMRV antigen is MAb 7C10 (see Wolff, L. et al., J. Virol. 43: 472-481 (1982)). An exemplary polyclonal antibody that binds an XMRV antigen is a polyclonal antibody against mouse xenotropic virus. Monoclonal and polyclonal antibodies can be generated using standard techniques known in the art (see generally, Carter (2006) Nat Rev Immunol. 6(5), 343-357; Teillaud (2005) Expert Opin Biol Ther. 5(Supp. 1) S15-27; Subramanian, ed. (2004) Antibodies: Volume 1: Production and Purification, Springer, ISBN 0306482452; Lo, ed. (2003) Antibody Engineering Methods and Protocols, Humana Press, ISBN 1588290921; Ausubel et al., ed. (2002) Short Protocols in Molecular Biology 5th Ed., Current Protocols, ISBN 0471250929; Brent et al., ed. (2003) Current Protocols in Molecular Biology, John Wiley & Sons Inc, ISBN 047150338X; Coligan (2005) Short Protocols in Immunology, John Wiley & Sons, ISBN 0471715786; Sidhu (2005) Phage Display In Biotechnology and Drug Discovery, CRC, ISBN-10: 0824754662). Aptamers can be produced against specific peptide motifs using standard techniques, such as, for example, those described in Ogawa, A., et al., Bioorg. Med. Chem. Lett. 14: 4001-4004, 2004; and Jayasena, S. D., Clinical Chemistry 45: 1628-1650, 1999. In various configurations, an aptamer can be, without limitation, an RNA aptamer, a DNA aptamer or a peptide aptamer.

As discussed herein, in a probe-based detection method, a supplied probe binds to a gammaretrovirus antigen from a sample. Such gammaretrovirus antigens can be according to antigens discussed below with respect to antigen-based methods. One of ordinary skill will recognize that such antigens can occur in a sample and be detected by a supplied probe, as described herein.

A molecular species which can contribute to or function as a probe can have a dissociation constant K_{d} for its binding target of less than about 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M or lower.

Detection of the presence, absence or quantity of a complex between a probe and an XMRV antigen can be according to any suitable means known in the art. A complex between a probe and an XMRV antigen can be detected according to one or more of an immunoprecipitation assay, an ELISA, a radioimmunoassay, a Western blot assay, or fluorescence detection methods, such as a flow cytometry assay or a fluorescence-activated cell sorting (FACS) assay.

A solid surface or substrate can be used in conjunction with the above described probe-based assay. A sample of a subject can be introduced to a substrate that binds, directly or indirectly, an XMRV antigen. After contacting the substrate with the sample, the substrate can be contacted with a probe that binds an XMRV antigen. Such approach can also include quantifying the amount of probe bound to the surface. The solid surface or probe can be, for example, an ELISA plate, a bead, a particle, a dip stick, a test strip, a membrane or a microarray. A probe or antigen can be adsorbed or attached to the support, e.g., through a covalent attachment. A cross-linking agent can be used to attach a probe or an antigen to a solid support.

A probe that binds an XMRV antigen can include a label. The label can be quantified as way of detecting the presence, absence or quantity of a complex between the probe and an XMRV antigen. Non-limiting examples of labels which can be used include moieties directly attached to a probe such as, without limitation, a radioisotope, a chromogen, a chromophore, a fluorophore, a fluorogen, an enzyme, such as horseradish peroxidase or alkaline phosphatase, quantum dots and resonance light scattering particles (Yguerabide, J., et al., Journal of Cellular Biochemistry Supplement 37: 71-81, 2001).

When a label is an enzyme, the enzyme can be any enzyme for which a substrate is available. Exemplary enzyme labels include, but are not limited to, a peroxidase (e.g., horseradish peroxidase); a phosphatase (e.g., alkaline phosphatase such as a secreted alkaline phosphatase), a galactosidase (e.g., a β-galactosidase), a β-glucoronidase, a luciferase (e.g., a firefly luciferase or a renilla luciferase), or a chloramphenicol acetyl transferase. In some configurations, a substrate can be a chromogen or a fluorogen, or can yield a chemiluminescent product. If the substrate is a chemiluminescent substrate, qualititative and/or quantitative detection of the enzyme can comprise measuring light produced as a product of a reaction between the substrate and the enzyme. For example, if the enzyme is an alkaline phosphatase, the substrate can be a chemiluminescent substrate such as CDP-Star® (Sigma-Aldrich Chemical Co., St. Louis, Mo.). In another example, if the enzyme is a luciferase, the substrate can be a luciferin. If the substrate is a chromogenic substrate, qualititative and/or quantitative detection of the enzyme can comprise visual assessment, and/or measuring optical absorbance of the reaction product, such as, without limitation, measuring absorbance at 400 nm when the enzyme is an alkaline phosphatase and the substrate is dinitrophenyl phosphate. If the substrate is a fluorogenic substrate, qualititative and/or quantitative detection of the enzyme can comprise visual assessment, and/or measuring fluorescent light intensity using a fluorometer.

When the label is a chromophore, the label can be any chromophore known to skilled artisans, such as, without limitation, a dichlorotriazine dye such as 1 Amino 4 [3 (4,6 dichlorotriazin 2 ylamino)4 sulfophenylamino]anthraquinone 2 sulfonic acid (Procion Blue MX R® (Fluka AG, Switzerland)). Such labels can be detected by methods known to skilled artisans, such as measurement of optical absorbance using a spectrophotometer.

When the label is a fluorophore, the label can be any fluorophore known to skilled artisans, such as, without limitation, a fluorescein, a rhodamine, an Alexa Fluor® (Invitrogen Corporation, Carlsbad, Calif.) a coumarin, an indocyanine or a quantum dot (Colton, H. M., et al., Toxicological Sciences 80: 183 192, 2004). In addition, in some configurations a fluorophore can be a fluorescent protein, such as a phycoerythrin or a fluorescent protein such as green fluorescent protein. Such fluorescent labels can be detected by methods known to skilled artisans, such as fluorescence microscopy or measurement of fluorescence using a fluorometer or a flow cytometry apparatus.

When the label is a radioisotope, the radioisotope can be any radioisotope known to skilled artisans, such as, without limitation, a ³²P, a ³³P, a ³⁵S, a ¹⁴C, an ¹²⁵I, an ¹³¹I or a ³H.

Other exemplary labels include, but are not limited to, biotin, a digoxygenin, and a peptide with an epitope. When the label is a probe binding target, the probe binding target can be any molecular target for a probe, such as, without limitation, a ligand to which a probe binds, such as, without limitation, an antigen which an antibody binds. In various configurations of these methods, a probe binding target can be, without limitation, a biotin, a digoxygenin, or a peptide, and a probe for the probe binding target can be, without limitation, an avidin, a streptavidin, an anti biotin antibody, an anti digoxygenin antibody, or a peptide antibody directed against a peptide. Accordingly, a label and a probe can be, without limitation, a) a biotin and an avidin, b) a biotin and a streptavidin, c) a biotin and an anti biotin antibody, d) a digoxygenin and an anti digoxygenin antibody, or e) a peptide and an antibody directed against the peptide.

In some configurations, a label can be bound indirectly to a probe, for example, a secondary antibody tagged with a fluorophore if the probe comprises a primary antibody. In some aspects, binding (or absence of binding) between a polypeptide to be identified and a binding pool can be detected using detection methods that do not require a separate label, such as, for example, surface plasmon resonance (SPR) and reflectometric interference spectroscopy (RIFS) (Gesellchen, F., et al., Methods in Molecular Biology 305: 17-46, 2005). In some configurations, a label can be one which can be removed, destroyed or quenched after it is detected, using techniques well known to skilled artisans. For example, in some configurations, a fluorophore can be bleached by intense irradiation with excitation wavelengths of light.

Detecting the presence, absence or quantity of a probe that binds an XMRV antigen can involve use of one or more secondary probes. A secondary probe can be contacted with a probe that binds an XMRV antigen or a complex between a probe and an XMRV antigen. The presence or amount of probe that binds an XMRV antigen can then be detected or quantified indirectly through the presence or quantity of the secondary probe. The presence or amount of an XMRV antigen can then be detected or quantified indirectly through the presence or quantity of the secondary probe.

A probe-based detection method can include selecting or modifying a treatment on the basis of the detection of the presence, absence or quantity of a probe that binds an XMRV antigen, or a secondary probe that bends thereto. For example, a therapeutically effective amount of an anti-retroviral compound can be administered to a subject upon detection of an XMRV in a sample from the subject. Therapeutic methods are discussed further below.

Antigen to Detect Gammaretroviral Antibodies in a Sample

Detecting, diagnosing, monitoring or managing an XMRV-related neuroimmune disease or XMRV-related lymphoma in a subject can include detection of XMRV based upon an gammaretrovirus antigen.

The present inventors have determined that the presence of antibodies against XMRV in a subject can be diagnostic for, or can aid in the diagnosis of, any XMRV-related disease, including any disease associated with XMRV infection, or for which XMRV infection is implicated or correlated. XMRV seroconversion is the development of detectable specific antibodies to XMRV in the blood serum as a result of infection by XMRV. Serology (the testing for antibodies) can be used to determine XMRV antibody positivity. Prior to XMRV seroconversion, a blood test can be seronegative for an XMRV antibody; after XMRV seroconversion, a blood test can be seropositive for XMRV antibody. Accordingly, detection or quantification of antibodies against XMRV in a subject can be used to diagnose an XMRV-related disease, monitor progress of an XMRV-related disease, or determine efficacy of a treatment of an XMRV-related disease in a subject.

An XMRV antibody generally includes an antibody that binds to XMRV or at least one molecular component thereof, such as, without limitation, a gag protein, an env protein, or a pol protein. An XMRV antibody can also be cross-reactive against a retrovirus in addition to XMRV or at least one antigenic component thereof, such as a gammaretrovirus. A gammaretrovirus to which a human XMRV antibody can be cross-reactive can be, without limitation, a spleen focus-forming virus, including a Friend spleen focus-forming virus, or a retrovirus within the Mammalian retrovirus group. This retrovirus group includes various murine leukemia-related retroviruses in addition to an XMRV, such as, without limitation, an Epicrionops marmoratus retrovirus, an Ichthyophis kohtaoensis retrovirus, an Osteolaemus tetraspis retrovirus, a Sericulus bakeri retrovirus, a Terdus iliacus retrovirus, a Tomistoma schlegelii retrovirus, and a Vipera berus retrovirus.

In an antigen-based method, a sample (which may contain or comprise gammaretroviral antibodies) can be obtained from a subject. The sample or subject can be as discussed above. A reaction mixture is formed with a sample from a subject and at least one gammaretrovirus antigen, such as an XMRV antigen. The reaction mixture can be exposed to conditions sufficient for formation of a complex between a gammaretrovirus antibody from the sample, if present, and a supplied XMRV antigen. The presence, absence or quantity of a complex between a sample antibody and an XMRV antigen can then be detected.

A gammaretrovirus antigen can be at least one of a Gag polypeptide, an Env polypeptide, a Pol polypeptide, or a fragment thereof. A gammaretrovirus antigen can include an XMRV polypeptide, or a portion thereof. A gammaretrovirus antigen can include a contiguous sequence of at least about 4 amino acids of a gammaretrovirus polypeptide, such as an XMRV polypeptide. For example, a gammaretrovirus antigen can include a contiguous sequence of at least about 5, at least about 6, at least about 7, at least about 8, at least about 9, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more amino acids of a gammaretrovirus polypeptide, such as an XMRV polypeptide. Such number of contiguous sequences can comprise a fragment of an antigen. A gammaretrovirus antigen can include a polypeptide having at least about 80%, at least about 85%, at least about 90%, at least about 95%, or at least about 99% sequence identity with a gammaretrovirus polypeptide, such as an XMRV Env polypeptide, an XMRV Gag polypeptide, or an XMRV Pol peptide, or fragment thereof. Such fragment can be of a number of contiguous amino acids as described above.

A gammaretrovirus antigen used to detect antibody that binds XMRV can be a fully- or partially-denatured polypeptide such as, for example, a fully- or partially-denatured XMRV polypeptide or a fully- or partially-denatured SFFV Env polypeptide, or can be a fully folded protein such as an XMRV Env protein or an SFFV Env protein.

An antigen that can be used to detect antibody that binds XMRV in a sample can be a polypeptide of a retrovirus taxonomically related to XMRV, such as a gammaretrovirus. A polypeptide of a gammaretrovirus of these embodiments can be, without limitation, an Env polypeptide of a retrovirus of the Mammalian virus group. Exemplary polypeptides include, but are not limited to, an Env polypeptide of a murine leukemia-related retrovirus, or an Env polypeptide of a gammaretrovirus such as a spleen focus-forming virus such as a Friend spleen focus-forming virus (SFFV), or a nonecotropic Murine Leukemia Virus such as a polytropic (Pmv) or a modified polytropic (Mmpv) (DeFreitas, E., et al., Proc. Nat'l. Acad. Sci. USA 88: 2922-2926, 1991).

An antigen that can be used to detect antibody against XMRV can be an antigen other than a Gag polypeptide of a gammaretrovirus such as XMRV or SFFV, such as a p30 Gag polypeptide, a p15 Gag polypeptide or a p10 Gag polypeptide.

Accordingly, various assays for detecting antibody against XMRV in a sample can include contacting a sample with a retrovirus polypeptide or a gammaretrovirus polypeptide such as an Env polypeptide, which can be, for example, an XMRV Env polypeptide or an SFFV Env polypeptide, and detecting binding of antibody in the sample to the polypeptide. The methods can include detecting binding of antibody in a sample to a polypeptide comprising a contiguous sequence of at least 4 amino acids, at least 5 amino acids, at least 6 amino acids, at least 7 amino acids, at least 8 amino acids, at least 9 amino acids, or at least 10 amino acids of a polypeptide of XMRV or a taxonomically related retrovirus such as a gammaretrovirus, such as, without limitation, an SFFV.

Exemplary sequences of XMRV polynucleotides and polypeptides are set forth in the Sequence Listing. GenBank Accession Number NC_{—}007815 (SEQ ID NO:1) is the complete genomic sequence of Xenotropic MuLV-related virus (XMRV) reference strain VP62. SEQ ID NO: 2 is an example of the gag-pol-pro polyprotein of XMRV. SEQ ID NO: 3 is an example of the Gag portion of the polyprotein of XMRV. SEQ ID NO: 4 is an example of a putative envelope protein of XMRV.

Exemplary sequences of gammaretroviral Env, Gag and Pol proteins are set forth in the sequence listings, and can be summarized as in Table 1.

TABLE 1
Sequences of gammaretroviral Env, Gag and Pol proteins

| SEQ ID | NCBI | | |
|---|---|---|---|
| NO: | Accession: | Virus/Isolate | Protein |
| 33 | ABB83226 | Xenotropic MuLV-related virus VP35 | putative envelope polyprotein |
| 34 | ABB83225 | Xenotropic MuLV-related virus VP35 | putative gag-pro-pol polyprotein |
| 35 | ABB83224 | Xenotropic MuLV-related virus VP35 | putative gag polyprotein |
| 36 | ABB83229 | Xenotropic MuLV-related virus VP42 | putative envelope polyprotein |
| 37 | ABB83228 | Xenotropic MuLV-related virus VP42 | putative gag-pro-pol polyprotein |
| 38 | ABB83227 | Xenotropic MuLV-related virus VP42 | putative gag polyprotein |
| 39 | YP_512363 | Xenotropic MuLV-related virus VP62 | putative envelope polyprotein |
| 40 | YP_512361 | Xenotropic MuLV-related virus VP62 | putative gag-pro-pol polyprotein |
| 41 | YP_512362 | Xenotropic MuLV-related virus VP62 | putative gag polyprotein |
| 42 | ABM47429 | Xenotropic MuLV-related virus VP62 | putative envelope glycoprotein |
| 43 | ABM47428 | Xenotropic MuLV-related virus VP62 | putative gag-pro-pol polyprotein |
| 44 | ABM47427 | Xenotropic MuLV-related virus VP62 | putative gag polyprotein |
| 45 | ABD49688 | Xenotropic MuLV-related virus VP62 | putative envelope polyprotein |
| 46 | ABD49687 | Xenotropic MuLV-related virus VP62 | putative gag-pro-pol polyprotein |
| 47 | ABD49686 | Xenotropic MuLV-related virus VP62 | putative gag polyprotein |
| 48 | P03393 | Friend spleen focus-forming virus (isolate | putative env polyprotein |
| | | 502) | |
| 49 | P03331 | Friend spleen focus-forming virus (isolate | putative gag/core polyprotein |
| | | 502) | |
| 50 | P31793 | Friend spleen focus-forming virus (strain | putative env polyprotein |
| | | BB6) | |
| 51 | P03394 | Friend spleen focus-forming virus (strain | putative env polyprotein |
| | | Lilly-Steeves) | |
| 52 | P03389 | Rauscher spleen focus-forming virus | putative env polyprotein |
| 53 | P03358 | Rauscher spleen focus-forming virus | putative pol polyprotein |
| 54 | AAA46506 | Rauscher spleen focus-forming virus | env polyprotein |
| 55 | AAA46505 | Rauscher spleen focus-forming virus | gp54 precursor peptide (env) |
| 56 | AAA46504 | Rauscher spleen focus-forming virus | polymerase (pol) |

Detection of the presence, absence or quantity of a complex between a sample gammaretrovirus antibody and a supplied gammaretrovirus antigen can be according to any suitable means known in the art. A complex between sample antibody and supplied antigen can be according to one or more of an immunoprecipitation assay, an ELISA, a radioimmunoassay, a Western blot assay, or fluorescence detection methods, such as a flow cytometry assay or a fluorescence-activated cell sorting (FACS) assays. Methods of detecting the presence, absence or quantity of antibody that binds XMRV can be other than a double antigen sandwich assay (Qiu, X., et al., J Med Virol. 80: 484-493, 2008).

A complex between a sample XMRV antibody and a supplied XMRV antigen can detected through use of a probe directed against a serum antibody. The reaction mixture can be exposed to conditions sufficient for formation of a probe/antibody complex. The presence, absence or quantity of the probe, probe/antibody complex, or probe/antibody/antigen complex can then be detected. The probe can be directed against human immunoglobulin. For example, probes directed against human immunoglobulin include, but are not limited to, an antibody, an antigen-binding fragment thereof, an aptamer or an avimer. Detection of the presence, absence or quantity of a complex between a probe/antibody complex or probe/antibody/antigen complex can be according to any suitable means known in the art, as discussed further above. A probe for use with a sample XMRV antibody and a supplied XMRV antigen can include a label. Probe labels and detection thereof can be as discussed above.

A competitive binding assay can be used to detect or quantify antibodies against a gammaretrovirus antigen, such as an XMRV antigen, in a sample. A competitive binding assay can include contacting, in the presence of a sample, at least one gammaretrovirus antigen with a probe that binds the at least one antigen, and detecting the extent of binding between such antigen and the probe. A reduction in the amount of binding between antigen and probe compared to a control can be indicative of the presence of antibody against XMRV in the sample. In various configurations, if the sample comprises antibody against an XMRV antigen, the quantity of complex will be less than that of a complex formed from a mixture comprising the XMRV antigen, the competitive probe, and a control sample not comprising antibody against the XMRV antigen. Hence, quantification of a complex comprising the at least one XMRV antigen and the competitive probe can be used to determine the presence, absence or quantity of antibody against XMRV in the sample. A competitive probe that binds a gammaretrovirus antigen can be according to probes discussed above in the context of a probe-based detection assay.

A gammaretrovirus antigen used to detect antibody that binds XMRV can be comprised by a eukaryotic cell ex vivo, such as a mammalian cell ex vivo or an insect cell ex vivo, or can be encoded by a polynucleotide and expressed in a microorganism, which can be a eukaryotic microorganism such as a yeast, or a prokaryotic microorganism such as an E. coli. A eukaryotic cell that expresses a polypeptide of the present teachings ex vivo can express the polypeptide on the cell surface or in the cytoplasm, or can secrete the polypeptide.

Some methods include providing at least one cell ex vivo that comprises an antigen that can be used to detect antibody against XMRV in a sample. The cell ex vivo can be a mammalian cell expressing a gammaretrovirus antigen, such as an XMRV antigen or an SFFV antigen ex vivo. The antigen can be, for example, an Env antigen of SFFV. Exemplary mammalian cells include a pro-B cell, such as a BaF3 cell a BaF3ER cell comprising an erythropoietin receptor. For example, a mammalian cell expressing at least one XMRV antigen can be a BaF3ER-SFFVEnV cell expressing Env protein of Friend spleen focus-forming virus (SFFV Env antigen).

A solid surface or substrate can be used in conjunction with the above described antigen-based assay. Some methods of the present teachings include providing a solid support comprising an antigen that can be used to detect antibody against XMRV in a sample. A gammaretrovirus antigen, such as an XMRV antigen, can be immobilized on a solid support. A solid support can be, for example, a bead, a particle, or an ELISA plate, and an antigen can be adsorbed or attached to the support, e.g., through a covalent attachment. A cross-linking agent can be used to attach an XMRV antigen to a solid support. A sample of a subject can be introduced to a substrate such that an XMRV antibody, if present in the sample, binds to an XMRV antigen of the solid support. The solid surface or substrate can be as discussed above.

Co-Culture Amplification

Detecting, diagnosing, monitoring or managing an XMRV-related neuroimmune disease or XMRV-related lymphoma in a subject can include amplification of XMRV from a sample from the subject. Amplification of XMRV can be accomplished through co-incubation of a sample from a subject with a cell type susceptible to infection with XMRV. The sample and subject can be as described above.

A cell type susceptible to infection with XMRV can be any cell type suitable for infection with a gammaretrovirus. A cell type susceptible to infection with XMRV can be, for example, an epithelial cell line, a fibroblast-like cell line, or a cell line derived from a cancer. A cell type susceptible to infection with XMRV can be derived from a mammal, for example from a human or a mouse. A cell type susceptible to infection with XMRV can express prostate specific antigen (PSA) or human prostatic acid phosphatase (hPAP). A cell type susceptible to infection with XMRV can have defects in the JAK-STAT pathway or the RNAse L pathway. A cell type susceptible to infection with XMRV can be, for example a LNCaP cell.

The sample from the subject can be activated prior to co-incubation with the cell type susceptible to infection with XMRV. The sample can be activated by treatment with, for example, IL-2 or phytohemagglutinin (PHA).

The sample from the subject can be centrifuged prior to incubation with the cell type susceptible to infection with XMRV. The centrifuged sample can be separated into its substituent parts. The sample, or a particular substituent of the sample, can be incubated with the cell type susceptible to infection with XMRV and then centrifuged. Centrifugation of the cell type susceptible to infection with XMRV and the sample or substituent of the sample can be comprised by the term “incubation.”

After the sample from the subject is incubated with the cell type susceptible to infection with XMRV, the sample can be separated from the cell type susceptible to XMRV. The cell type susceptible to infection with XMRV can be allowed to replicate, either before or after the sample is separated from the replicating cells.

After the sample is incubated with the cell type susceptible to infection with XMRV, the presence, absence or amount of XMRV present can be determined. Determining the presence, absence or amount of XMRV can be according to any method disclosed herein, including but not limited to, detection of at least one XMRV antigen, detection of at least one XMRV polypeptide or detection of at least one XMRV polynucleotide.

Hybridization

Detecting, diagnosing, monitoring or managing an XMRV-related neuroimmune disease or XMRV-related lymphoma in a subject can include XMRV nucleic acid detection methods. Detection of XMRV can be based upon a hybridization assay.

In an hybridization-based method, a reaction mixture can be formed with a biological sample from a subject and a nucleobase polymer that hybridizes to XMRV. The sample or subject can be as discussed above. The reaction mixture can be exposed to conditions sufficient for hybridization to occur between an XMRV nucleic acid or the complement thereof (if present in the sample) and the nucleobase polymer that hybridizes to XMRV. Conditions sufficient for hybridization to occur include highly stringent hybridization conditions, as discussed below. The hybridization conditions can be of sufficient stringency to effect hybridization between the nucleobase polymer and an XMRV sequence but not other viruses. After reaction of the mixture, the presence, absence or quantity of a hybridization complex between an XMRV nucleic acid, or complement thereof, and the nucleobase polymer that hybridizes to XMRV can be detected. Detection of the hybridization complex can be according to any suitable method know in the art.

A nucleobase polymer that hybridizes to XMRV can be a nucleic acid or a nucleic acid analogue. A nucleic acid that hybridizes to XMRV can be a DNA. A nucleic acid that hybridizes to XMRV can be an RNA. A nucleic acid analogue that hybridizes to XMRV can be a peptide-nucleic acid.

A nucleobase polymer that hybridizes to XMRV can be a labeled nucleobase polymer. Such a label can include, but is not limited to, one or more of a radioisotope, an enzyme, a hapten, a fluorogen, a fluorophore, a chromogen, or a chromophore.

A nucleobase polymer that hybridizes to XMRV can have at least about 10 contiguous nucleotides of an XMRV nucleic acid or a complement thereof. For example, a nucleobase polymer (e.g., polynucldeotide) that hybridizes to XMRV can have at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more contiguous nucleotides of an XMRV nucleic acid or a complement thereof.

A nucleobase polymer that hybridizes to XMRV can be the complement of a sequence having at least about 70% sequence identity with a sequence of an XMRV nucleic acid or a complement thereof. For example, nucleobase polymer (e.g., polynucldeotide) that hybridizes to XMRV can have be the complement of a sequence having at least about 75%, at least about 80%, at least about 85%, at least about 90%, at least about 95%, at least about 96%, at least about 97%, at least about 98%, or at least about 99% sequence identity with a sequence of an XMRV nucleic acid or a complement thereof. A nucleobase polymer (e.g., polynucldeotide) that hybridizes to XMRV can be the complement of a sequence having 100% sequence identity with a sequence of an XMRV nucleic acid or a complement thereof. Such nucleobase polymers having any of the above sequence identity with XMRV or a complement thereof can be of a size described above (e.g., at least about 10 nucleotides, at least about 15 nucleotides, etc.).

Detection of the presence, absence or quantity of a hybridization complex between a nucleobase polymer and an XMRV nucleic acid or complement thereof can be according to any suitable means known in the art. Detection of a hybridization complex can be according to one or more of a Southern hybridization assay, a northern hybridization assay, a dot-blot hybridization assay, a slot-blot hybridization assay, a Polymerase Chain Reaction (PCR) assay, or a flow cytometry assay. For example, detection of a hybridization complex can be according to a quantitative real time PCR assay. A hybridization assay can include use of one or more primers of SEQ ID NOS: 5-20.

A hybridization-based detection method can include selecting or modifying a treatment on the basis of the detection of the presence, absence or quantity of a hybridization complex between an XMRV nucleic acid, or complement thereof, and the nucleobase polymer that hybridizes to XMRV. For example, a therapeutically effective amount of an anti-retroviral compound can be administered to a subject upon detection of an XMRV in a sample from the subject. Therapeutic methods are discussed further below.

Screening

One aspect provides a method for screening for an anti-viral agent, such as an anti-XMRV agent. The method can be in conjunction with methods for detecting an XMRV-related neuroimmune disease or XMRV-related lymphoma, as described above.

In a screening method, a candidate compound can be contacted with cells, such as peripheral blood mononuclear cells, infected with XMRV. Virus production by the peripheral blood mononuclear cells infected with XMRV can be monitored. Any method suitable for detecting XMRV levels resultant from administration of the candidate substance can be employed, such as those detection methods discussed above. A candidate agent that reduces or eliminates detectable virus production by the peripheral blood mononuclear cells can be selected as an anti-XMRV agent.

The screening method can include contacting a candidate compound and a first group of peripheral blood mononuclear cells infected with XMRV and contacting the candidate compound and a second group of peripheral blood mononuclear cells not infected with XMRV. Cell death in the XMRV-infected cells and the non-XMRV-infected cells can be quantified. A candidate compound can be selected as an anti-retroviral agent against XMRV infection if cell death is more extensive in the XMRV-infected cells compared to the non-XMRV-infected cells. For example, a candidate compound can be selected as an anti-retroviral agent against XMRV infection if cell death is at least about 10% increased in the XMRV-infected cells compared to the non-XMRV-infected cells. As another example, a candidate compound can be selected as an anti-retroviral agent against XMRV infection if cell death is at least about 25%, about 50%, about 75%, about 100%, about 200%, about 300%, about 400%, about 500%, about 1000%, or more increased in the XMRV-infected cells compared to the non-XMRV-infected cells.

Peripheral blood mononuclear cells for use in screening methods can be as described above and include an in vitro culture of peripheral blood mononuclear cells infected with XMRV. Exemplary peripheral blood mononuclear cells infected with XMRV for use in screening methods include, but are not limited to cells from one or more of WPI 1282, WPI 2119 and WPI 2767 cell lines.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 Daltons mw, or less than about 1000 Daltons mw, or less than about 800 Daltons mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Preferably, initial screening is performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present invention.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Cells

One aspect provides a cell in vitro or a cell line in vitro harboring XMRV. The in vitro cell line can include peripheral blood mononuclear cells harboring XMRV. Peripheral blood mononuclear cells can include, but are not limited to, B cells, T cells, macrophages, dendritic cells, monocytes, fibroblasts, haematopoetic cells and endothelial cells. A cell line or culture can include one or more of such cell types. For example, the in vitro cell line can include B cells and T cells harboring XMRV. A cell of the in vitro cell line can produce XMRV. The peripheral blood mononuclear cells can be contacted with a cytokine or interleukin, such as, without limitation, one or more of EGF, TGF-β, FGF and IL-10. A cell line can be a cell line to be deposited with the American Type Culture Collection under ATCC accession numbers PTA-10139 (ERV and MRV infected P. Blood Hu MCL, male, WPI-1282); PTA-10140 (XMRV infected Hu B-cell line, BM, CFS, female, WPI-2119); or PTA-10141 (EBV and XMRV infected Hu B cell line, B1, male, WPI-2767). Cell lines harboring XMRV can be used to study a neuroimmune disease or lymphoma, such as MCL in vitro (see e.g., Drexler, H., et al., Leukemia Research 26: 781787, 2002), or can be used to screen for compounds for treating a neuroimmune disease, a lymphoma, or an XMRV infection.

Also provide is an in vitro cell culture. An in vitro cell culture can include an in vitro cell line, as discussed above, and a cell culture medium. The cell culture medium can include, for example, an cytokine or interleukin. For example, the cell culture medium can include EGF, TGF-β, FGF or IL-10. As another example, the cell culture medium can include interleukin IL-10. A cell culture can include cell that produce XMRV in vitro. Such cells can be lymphoma cells (e.g., mantle cell lymphoma cells). Such cells can be lymphoblastoid cells.

An in vitro cell line can be formed from peripheral blood mononuclear cells from a subject infected with XMRV. A reaction mixture can be formed in vitro from peripheral blood mononuclear cells from a subject infected with XMRV, a cell culture medium, and a cytokine or interleukin. An in vitro cell line can be formed by innoculating peripheral blood mononuclear cells with XMRV in vitro. Peripheral blood mononuclear cells can be as discussed above. Cell culture medium, cytokines and interleukins can be as discussed above.

Vaccine and Conferred Immunity

One aspect provides a vaccine, and use thereof, for conferring immunity against an XMRV-related disease or condition. The vaccine can include an XMRV antigen, or a nucleic acid vector encoding an XMRV antigen. For example, a nucleic acid vector can include a promoter operably linked to a nucleic acid encoding an XMRV antigen, or a portion thereof having at least 4, at least 5, at least 6, at least 7, at least 8, at least 9, or at least 10 contiguous amino acids of an XMRV polypeptide. A vector can be, for example and without limitation, a plasmid, a bacteriophage, an insect virus, or a mammalian virus, such as, for example an adeno-associated virus such as AAV5. The promoter can be heterologous to XMRV. As another example, an XMRV antigen of the vaccine can include an epitope recognized by the 7C10 antibody. A vaccine can include an XMRV polypeptide, or portion thereof, as described herein, such as, without limitation, an XMRV envelope protein. The vaccine can include a live virus, such as an attenuated XMRV.

The vaccine can further include a pharmaceutically acceptable carrier, an adjuvant, or a combination thereof, in accordance with well known principles of vaccine design. Pharmaceutical formulation can be as discussed below. A vaccine of the present teachings can be administered to a subject using any method known to skilled artisans, as discussed further below.

The vaccine including an XMRV antigen, or a nucleic acid vector encoding an XMRV antigen, can confer immunity against XMRV-related Chronic Fatigue Syndrome (CFS), XMRV-related fibromyalgia, XMRV-related Multiple Sclerosis (MS), XMRV-related Parkinson's Disease, XMRV-related Amyotrophic Lateral Sclerosis (ALS), XMRV-related autism spectrum disorder (ASD), XMRV-related chronic lyme disease, XMRV-related Mantle Cell Lymphoma (MCL), or XMRV-related Chronic Lymphocytic Leukemia lymphoma (CLL).

Also provided is a method of conferring immunity against an XMRV-related disease or condition. The method of conferring immunity can include administering an effective amount of a vaccine described above to a subject in need thereof. Need of a subject can be as discussed above. XMRV-related disease or condition can be as discussed above.

Also provided is a method of producing a vaccine against an XMRV-related disease or condition. Such vaccines can be produced in accordance with established methods known to skilled artisans, such as serial passage adaptation (see e.g., Sabin, A. B., Ann. N.Y. Acad. Sci. 61: 924-938, 1955) or through genetic engineering techniques (e.g., through alteration of codons). A vaccine can be produced using XMRV produced by cells of the cell lines described herein. The method of producing a vaccine can include isolating and inactivating XMRV. The XMRV can be an attenuated XMRV. The method of producing a vaccine can include isolating an XMRV antigen. XMRV or XMRV antigen can be obtained from an in vitro cell culture. The method of producing a vaccine can include expressing an XMRV antigen in vitro. An XMRV antigen can be produced in a eukaryotic or prokaryotic cell, such as, without limitation, a yeast cell, a mammalian cell such as a murine cell or a human cell, an avian cell, an insect cell or a bacterial cell such as an E. coli. In these configurations, a cell expressing an XMRV antigen can include a nucleic acid vector such as a plasmid or virus comprising a promoter operably linked to a nucleic acid sequence encoding an XMRV antigen or portion thereof.

A vaccine can be produced by isolating an XMRV antigen, such as the XMRV antigen recognized by an antibody that recognizes and binds XMRV, such as monoclonal antibody 7C10. An exemplary XMRV antigen is an XMRV envelope polypeptide. An XMRV antigen can be a polypeptide having an epitope recognized by an antibody that binds XMRV (e.g, monoclonal antibody 7C10). An XMRV antigen, such as the epitope recognized by monoclonal antibody 7C10, can be isolated from XMRV, from an XMRV-infected cell line such as WPI 1282, WPI 2119 or WPI 2767 cell lines described herein, or from a cell comprising a nucleic acid vector, wherein the vector comprises a promoter operably linked to sequence encoding an XMRV polypeptide or portion thereof, such as an XMRV antigen comprising the epitope recognized by monoclonal antibody 7C10. The vaccine produced can be as discussed above.

Compositions and Formulation

Also provided are anti-retroviral agents for treating an XMRV-related disease or disorder. For example, anti-retroviral agents can be used to treat an XMRV-related neuroimmune disease or an XMRV-related lymphoma. An anti-retroviral agent can be an anti-retroviral compound or pharmaceutical composition including an anti-retroviral compound. Examples of anti-retroviral agents that can be used to manage or treat an XMRV-related neuroimmune disease or an XMRV-related lymphoma include, but are not limited to, acyclovir, penciclovir (famciclovir), gancyclovir (ganciclovir), deoxyguanosine, foscarnet, idoxuridine, trifluorothymidine, vidarabine, sorivudine, zidovudine (AZT, ZVD, azidothyidine, e.g., Retrovir), didanosine (ddI, e.g., Videx and Videx EC), zalcitabine (ddC, dideoxycytidine, e.g., Hivid), lamivudine (3TC, e.g., Epivir), stavudine (d4T, e.g., Zerit and Zerit XR), abacavir (ABC, e.g., Ziagen), emtricitabine (FTC, e.g., Emtriva (formerly Coviracil)), entecavir (INN, e.g., Baraclude), apricitabine (ATC), tenofovir (tenofovir disoproxil fumarate, e.g., Viread), adefovir (bis-POM PMPA, e.g., Preveon and Hepsera), multinucleoside resistance A, multinucleoside resistance B, nevirapine (e.g., Viramune), delavirdine (e.g., Rescriptor), efavirenz (e.g., Sustiva and Stocrin), etravirine (e.g., Intelence), adefovir dipivoxil, indinavir, ritonavir (e.g., Norvir), saquinavir (e.g., Fortovase, Invirase), nelfinavir (e.g., Viracept), agenerase, lopinavir (e.g., Kaletra), atasanavir (e.g., Reyataz), fosamprenavir (e.g., Lexiva, Telzir), tipranavir (e.g., Aptivus), darunavir (e.g., Prezista), amprenavir, deoxycytosine triphosphate, lamivudine triphosphate, emticitabine triphosphate, adefovir diphosphate, penciclovir triphosphate, lobucavir triphosphate, amantadine, rimantadine, zanamivir and oseltamivir, raltegravir (e.g., Isentress), elvitegravir (e.g., GS 9137 or JTK-303), MK-2048, maraviroc (e.g., Celsentri), enfuvirtide (e.g., Fuzeon), TNX-355, PRO140, BMS-488043, plerixafor, epigallocatechin gallate, vicriviroc, aplaviroc, b12 (an antibody against HIV found in some long-term nonprogressors), griffithsin, DCM205, bevirimat, and vivecon. For example, one or more of AZT and cidofovir can be used to manage or treat an XMRV-related neuroimmune disease or an XMRV-related lymphoma. As another example, an interferon (e.g., interferon-β) can be used to manage or treat an XMRV-related neuroimmune disease or an XMRV-related lymphoma.

As another example, an agent for treating an XMRV-related disease or disorder can target the immune response to the retrovirus, such as NfkB inhibitors and monoclonal antibodies targeting virally infected cells including, but not limited to, Rituxan and Velcade.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, preferably in purified form, together with a suitable amount of carrier so as to provide the form for proper administration to the subject.

The formulation should suit the mode of administration. The agents of use with the current invention can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating an XMRV-related neuroimmune disease or an XMRV-related lymphoma in a subject in need administration of a therapeutically effective amount of an anti-retroviral agent. Such treatment method can reduce or eliminate XMRV replicating. Treatment methods can be in conjunction with detecting, diagnosing, monitoring, or managing an XMRV-related neuroimmune disease, an XMRV-related lymphoma, or both.

Methods described herein are generally performed on a subject in need thereof. A subject in need of treatment as described herein can be a subject having, diagnosed with, suspected of having, or at risk for developing a neuroimmune disease or a lymphoma, such as an XMRV-related neuroimmune disease or an XMRV-related lymphoma. A subject can be considered at risk of developing a neuroimmune disease or lymphoma where, without limitation, an individual with a familial history of a neuroimmune disease or a lymphoma, such as an XMRV-related neuroimmune disease or an XMRV-related lymphoma, or an individual resides or has resided in a region comprising a cluster of individuals with such diseases or disorders.

Examples of an XMRV-related lymphoma include, but are not limited to an XMRV-related Mantle Cell Lymphoma (MCL) and a Chronic Lymphocytic Leukemia lymphoma (CLL). Examples of an XMRV-related neuroimmune disease include, but are not limited to Chronic Fatigue Syndrome (CFS), fibromyalgia, Multiple Sclerosis (MS), Parkinson's Disease, Amyotrophic Lateral Sclerosis (ALS), autism spectrum disorder (ASD), and chronic lyme disease. For example, CFS can be treated in a subject by administering a therapeutically effective amount of an anti-retroviral compound. As another example, MS, such as Atypical Multiple Sclerosis, can be treated in a subject by administering a therapeutically effective amount of an anti-retroviral compound or pharmaceutical composition including an anti-retroviral compound.

A determination of the need for treatment will typically be assessed by a history and physical exam consistent with the disease or condition at issue. For example, a subject can be tested for the presence of an XMRV where the subject exhibits signs or symptoms of a neuroimmune disease or a lymphoma. Diagnosis of the various conditions treatable by the methods described herein is within the skill of the art. The subject can be an animal subject, preferably a mammal, more preferably horses, cows, dogs, cats, sheep, pigs, mice, rats, monkeys, guinea pigs, and chickens, and most preferably a human.

For example, a subject can be one which fulfills the 1994 CDC Fukuda Criteria for CFS (Fukuda et al., Ann Intern Med 1994; 121: 953-9); the 2003 Canadian Consensus Criteria (CCC) for ME/CFS (Carruthers et al, J Chronic Fatigue Syndrome 2003; 11:1-12; Jason et al., J Chronic Fatigue S 2004; 12:37-52), or both the Fukuda and CCC criteria. The CCC requires post-exertional malaise, which many clinicians believe is the sine qua non of ME/CFS. In contrast, the Fukuda and 1991 Oxford Criteria do not require exercise intolerance for a diagnosis of ME/CFS. The CCC further requires that subjects exhibit post exertional fatigue, unrefreshing sleep, neurological/cognitive manifestations and pain, rather than these being optional symptoms.

As another example, the subject can be an animal, such as a laboratory animal that can serve as a model system for investigating a neuroimmune disease or MCL (see e.g., Chen, R. et al., Neurochemical Research 33: 1759-1767, 2008; Kumar, A., et al., Fundam. Clin. Pharmacol. Epub ahead of print, Jan. 10, 2009; Gupta, A., et al., Immunobiology 214: 33-39, 2009; Singh, A., et al., Indian J. Exp. Biol. 40: 1240-1244, 2002; Ford, R. J., et al. Blood 109: 4899-4906, 2007; Smith, M. R., et al., Leukemia 20: 891-893, 2006; Bryant, J., et al., Lab. Invest. 80: 557-573, 2000; M'kacher, R., et al., Cancer Genet Cytogenet. 143: 32-38, 2003).

An effective amount of an anti-retroviral agent described herein is generally that which can reduce or eliminate XMRV replication or reduce or eliminate signs or symptoms of a neuroimmune disease or a lymphoma. An anti-retroviral agent can be an anti-retroviral compound or pharmaceutical composition including an anti-retroviral compound. Examples of anti-retroviral agents that can be used to manage or treat an XMRV-related neuroimmune disease or an XMRV-related lymphoma include, but are not limited to, acyclovir, penciclovir (famciclovir), gancyclovir (ganciclovir), deoxyguanosine, foscarnet, idoxuridine, trifluorothymidine, vidarabine, sorivudine, zidovudine (AZT, ZVD, azidothyidine, e.g., Retrovir), didanosine (ddI, e.g., Videx and Videx EC), zalcitabine (ddC, dideoxycytidine, e.g., Hivid), lamivudine (3TC, e.g., Epivir), stavudine (d4T, e.g., Zerit and Zerit XR), abacavir (ABC, e.g., Ziagen), emtricitabine (FTC, e.g., Emtriva (formerly Coviracil)), entecavir (INN, e.g., Baraclude), apricitabine (ATC), tenofovir (tenofovir disoproxil fumarate, e.g., Viread), adefovir (bis-POM PMPA, e.g., Preveon and Hepsera), multinucleoside resistance A, multinucleoside resistance B, nevirapine (e.g., Viramune), delavirdine (e.g., Rescriptor), efavirenz (e.g., Sustiva and Stocrin), etravirine (e.g., Intelence), adefovir dipivoxil, indinavir, ritonavir (e.g., Norvir), saquinavir (e.g., Fortovase, Invirase), nelfinavir (e.g., Viracept), agenerase, lopinavir (e.g., Kaletra), atasanavir (e.g., Reyataz), fosamprenavir (e.g., Lexiva, Telzir), tipranavir (e.g., Aptivus), darunavir (e.g., Prezista), amprenavir, deoxycytosine triphosphate, lamivudine triphosphate, emticitabine triphosphate, adefovir diphosphate, penciclovir triphosphate, lobucavir triphosphate, amantadine, rimantadine, zanamivir and oseltamivir, raltegravir (e.g., Isentress), elvitegravir (e.g., GS 9137 or JTK-303), MK-2048, maraviroc (e.g., Celsentri), enfuvirtide (e.g., Fuzeon), TNX-355, PRO140, BMS-488043, plerixafor, epigallocatechin gallate, vicriviroc, aplaviroc, b12 (an antibody against HIV found in some long-term nonprogressors), griffithsin, DCM205, bevirimat, and vivecon. For example, one or more of AZT and cidofovir can be used to manage or treat an XMRV-related neuroimmune disease or an XMRV-related lymphoma. As another example, an interferon (e.g., interferon-β) can be used to manage or treat an XMRV-related neuroimmune disease or an XMRV-related lymphoma.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an anti-retroviral agent can be employed in pure form or, where such forms exist, in pharmaceutically acceptable salt form and with or without a pharmaceutically acceptable excipient. For example, the compounds of the invention can be administered, at a reasonable benefit/risk ratio applicable to any medical treatment, in a sufficient amount to reduce or eliminate XMRV replication or reduce or eliminate signs or symptoms of a neuroimmune disease or a lymphoma.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present invention will be decided by an attending physician within the scope of sound medical judgment.

Administration of an anti-retroviral agent can occur as a single event or over a time course of treatment. For example, an anti-retroviral agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for an XMRV-related neuroimmune disease or an XMRV-related lymphoma.

An anti-retroviral agent can be administered simultaneously or sequentially with another agent, such as an antibiotic, an antiinflammatory, or another agent. For example, an anti-retroviral agent can be administered simultaneously with another agent, such as an antibiotic or an antiinflammatory. Simultaneous administration can occur through administration of separate compositions, each containing one or more of an anti-retroviral agent, an antibiotic, an anti-inflammatory, or another agent. Simultaneous administration can occur through administration of one composition containing two or more of an anti-vial agent, an antibiotic, an antiinflammatory, or another agent. An anti-retroviral agent can be administered sequentially with an antibiotic, an antiinflammatory, or another agent. For example, an anti-retroviral agent can be administered before or after administration of an antibiotic, an antiinflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the invention.

Delivery systems may include, for example, an infusion pump, which may be used to administer the agent or composition in a manner similar to that used for delivering insulin or chemotherapy to specific organs or tumors. Typically, using such a system, an agent or composition is administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart ploymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Molecular Engineering

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art. Generally, conservative substitutions can be made at any position so long as the required activity is retained.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀ [Na⁺])+0.41(fraction G/C content)−0.63(% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem. Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Additional methods and compositions described herein utilize laboratory techniques set forth in PCT Patent Application PCT/US2006/013167, published as International Publication Number WO2006/110589, “Gammaretrovirus Associated With Cancer” Silverman, R., et al., inventors.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to probes, antigens, primers, reaction mixture components, anti-retroviral agents, etc. Such packaging of the components separately can, if desired, be presented in a pack or dispenser device which may contain one or more unit dosage forms or components for one or more reactions. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Definitions and methods described herein are provided to better define the present invention and to guide those of ordinary skill in the art in the practice of the present invention. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the invention are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Blood Preparation and Nucleic Acid Isolation

Whole blood was drawn from subjects by venipuncture using standardized phlebotomy procedures into 8-mL greencapped Vacutainers containing the anti-coagulant sodium heparin (Becton Dickinson). Plasma was collected by centrifugation, aspirated and stored at −80° C. for later use. The plasma was replaced with PBS and the blood resuspended and further diluted with an equal volume of PBS. PBMCs were isolated by layering the diluted blood onto Ficoll-Paque PLUS (GE Healthcare), centrifuging for 22 min at 800 g, aspirating the PBMC layer and washing it once in PBS. The PBMCs (approximately 2×10⁷ cells) were centrifuged at 500 g for 7 min and either stored as frozen unactivated cells in 90% FBS and 10% DMSO at −80° C. for further culture and analysis or resuspended in TRIzol (Invitrogen) and stored at −80° C. for DNA and RNA extraction and analysis.

DNA was isolated from TRIzol according the to manufacturer's protocol and also isolated from frozen PBMC pellets using the QIAamp DNA Mini purification kit (QIAGEN) according to the manufacturer's protocol and the final DNA was resuspended in RNase/DNase free water and quantified using the Quant-iT Pico Green dsDNA Kit (Invitrogen). RNA was isolated from TRIzol according to the manufacturer's protocol and quantified using the Quant-iT Ribo Green RNA kit (Invitrogen). cDNA was made from RNA using the iScript Select cDNA synthesis kit (Bio-Rad) according to the manufacturer's protocol.

### Example 2

### PCR

To avoid potential problems with laboratory DNA contamination, nested PCR was performed with separate reagents in a separate laboratory room designated to be free of high copy amplicon or plasmid DNA. Negative controls in the absence of added DNA were included in every experiment. Identification of XMRV gag and env genes was performed by PCR in separate reactions.

Reactions were performed as follows: 100 to 250 ng DNA, 2 μL of 25 mM MgCl2, 25 μL of HotStart-IT FideliTaq Master Mix (USB Corporation), 0.75 μL of each of 20 μM forward and reverse oligonucleotide primers in reaction volumes of 50 μL. For identification of gag, 419F (5′-ATCAGTTAACCTACCCGAGTCGGAC-3′) (SEQ ID NO: 5) and 1154R (5′-GCCGCCTCTTCTTCATTGTTCTC-3′) (SEQ ID NO: 6) were used as forward and reverse primers. For env, 5922F (5′-GCTAATGCTACCTCCCTCCTGG-3′) (SEQ ID NO: 7) and 6273R (5′-GGAGCCCACTGAGGAATCAAAACAGG-3′) (SEQ ID NO: 8) were used. For both gag and env PCR, 94° C. for 4 min initial denaturation was performed for every reaction followed by 94° C. for 30 seconds, 57° C. for 30 seconds and 72° C. for 1 minute. The cycle was repeated 45 times followed by final extension at 72° C. for 2 minutes. Six microliters of each reaction product was loaded onto 2% agarose gels in TBE buffer with 1 kb+DNA ladder (Invitrogen) as markers. PCR products were purified using Wizard SV Gel and PCR Clean-Up kit (Promega) and sequenced.

PCR amplification for sequencing full-length XMRV genomes was performed on DNA amplified by nested or semi-nested PCR from overlapping regions from PBMC DNA. For 5′ end amplification of R-U5 region, 4F (5′-CCAGTCATCCGATAGACTGAGTCGC-3′) (SEQ ID NO: 9) and 1154R was used for first round and 4F and 770R (5′-TACCATCCTGAGGCCATCCTACATTG-3′) (SEQ ID NO: 10) was used for second round. For regions including gag-pro and partial pol, 350F (5′-GAGTTCGTATTCCCGGCCGCAGC-3′) (SEQ ID NO: 11) and 5135R (5′-CCTGCGGCATTCCAAATCTCG-3′) (SEQ ID NO: 12) was used for first round followed by second round with 419F and 4789R (5′-GGGTGAGTCTGTGTAGGGAGTCTAA-3′). (SEQ ID NO: 13) For regions including partial pol and env region, 4166F (5′-CAAGAAGGACAACGGAGAGCTGGAG-3′) (SEQ ID NO: 14) and 7622R (5′-GGCCTGCACTACCGAAAT TCTGTC-3′) (SEQ ID NO: 15) were used for first round followed by 4672F (5′GAGCCACCTACAATCAGACAAAAGGAT-3′) (SEQ ID NO: 16) and 7590R (5′-CTGGACCAAGCGGTTGAGAATACAG-3′) (SEQ ID NO: 17) for second round. For the 3′ end including the U3-R region, 7472F (5′-TCAGGACAAGGGTGGTTTGAG-3′) (SEQ ID NO: 18) and 8182R (5′-CAAACAGCAAAAGGCTTTATTGG-3′) (SEQ ID NO: 19) were used for first round followed by 7472F and 8147R (5′-CCGGGCGACTCAGTCTATC-3′) (SEQ ID NO: 20) for second round. The reaction mixtures and conditions were as described above except for the following: For larger fragments, extension was done at 68° C. for 10 min instead of 72° C. All second round PCR products were column purified as mentioned above and overlapping sequences were determined with internal primers. Nested RT-PCR for gag sequences was done as described with modifications. GAG-O-R primer was used for 1st strand synthesis; cycle conditions were 52° C. annealing, for 35 cycles. For second round PCR, annealing was at 54° C. for 35 cycles.

### Example 3

### Cells

Isolation, separation and culture of primary cells.

Leukopaks of peripheral blood from healthy donors were collected according to a NIH approved IRB #99-CC-0168 protocol. Subjects' peripheral blood and plasma samples were from frozen banked samples obtained under NIH exempt status. Mononuclear leukocytes from both normal and subjects' cells were isolated by Ficoll-Hypaque gradient centrifugation. The light density fraction (buffy coat) was collected, washed twice with PBS. PBMC were activated by 1 μg/mL PHA (Abbott Diagnostics) and after 72 hours the cells were cultured with 20 units/mL of IL-2 (Zeptometrix) and subcultured every 3-5 days. For isolation of CD4+ T cells, CD8, CD11b, CD14, CD19, CD33 and CD56 positive cells were removed using magnetic activated cell sorting (MACS) methods according to manufacturer's instructions (Miltenyi Biotec, Inc.). After isolation, the CD3+, CD4 T cells (>95% pure) were cultured in RPMI-1640 medium supplemented with 10% fetal calf serum (FCS), 2 mM glutamine, 1 mM sodium pyruvate and antibiotics. CD4+ T cells were activated by culturing with 20 units/mL of IL-2 and 1 μg/mL PHA.

In Vitro Expansion of Primary B-Cells.

NIH 3T3 cells transduced with a retroviral vector expressing CD40L were maintained in Dulbecco's Modified Eagle's Medium (DMEM) (Invitrogen) supplemented with 10% calf serum (CS) (Lonza) and 1% Pennicillin, Streptomycin and L-Glutamine (Invitrogen) at 37° C. with 5% CO₂. To stimulate B cell expansion, ˜3.5×10⁶ NIH3T3-CD40L cells were trypsinized (0.25% trypsin with EDTA) (Invitrogen), resuspended in 3 mL medium and irradiated with an absorbed radiation dose (rad) of 9600 using a Cesium₁₃₇ Irradiator. Cells plus 7 mL medium were added to a T75 cell culture flask (Corning) and allowed to adhere (2-3 h) to the flask surface (optimal density ˜50%). CD19+ B cells were isolated from PBMCs using immunomagnetic bead technology (Miltenyi Biotec). CD19+ cells were separated from 10⁸ freshly isolated PBMCs by positive selection with a purity of >95% according to the manufacturer's protocol. After magnetic separation, CD19+ B cells were added to an irradiated NIH3T3-CD40L monolayer and incubated at 37° C. with 5% CO₂. Cultures were monitored for B cell proliferation and split 1:5 every 72-96 h onto freshly irradiated NIH 3T3-CD40L monolayer. CD19+ primary B cells were cultured and expanded in primary B cell expansion media: Iscove's Modified Dulbecco's Medium (IMDM) (Invitrogen)+10% FCS (Atlanta Biologicals), 1% Penicillin, Streptomycin and L-Glutamine (Invitrogen), 40 ng/mL interleukin 4 (IL-4) (PeproTech, Inc.), 50 μg/mL holo-transferrin (Sigma) and 5 μg/mL insulin (Invitrogen).

Cell Culture and Reagents.

Raji, SupT1 and LNCaP were obtained from American Type Culture Collection (ATCC). The cells were maintained in RPMI-1640 supplemented with L-glutamine (2 mM), penicillin (100 U/mL), streptomycin (100 ng/mL), and FCS (10%) and subcultured 1:5 every 4-5 days.

### Example 4

### Flow Cytometry for Viral Proteins

Adherent cells were incubated in trypsin for 10 minutes at 37° C. After additional washes, adherent and suspension cells were incubated for 15 min at RT in 1 mL of paraformaldehyde (4% w/v in PBS), washed in permeabilization wash buffer (0.5% saponin 0.1%, sodium azide, 2% human AB sera in PBS) (PWB), and resuspended in 300 μL of permeabilization buffer (PBS with 2.5% saponin) (PB).

After incubating at 22° C. for 20 min, 5 mL of human AB sera and either a rat anti-MuLV p30, rat anti-SFFV gp55 Env, goat anti-Rauscher gp 70 Env, p30 Gag, and p10 Gag or the appropriate isotype control (anti-rat IgG, rat hybridoma supernatant, or preimmune goat serum) were added, and the cells were incubated at 4° C. for an additional 30 min. Cells were then washed in PWB, resuspended in 100 μL of PB with 3 μL (0.6 μg) of FITC-conjugated goat anti-rat IgG or rabbit anti-goat antibody (BD PharMingen), and incubated for 20 min at 4° C.

The efficiency of permeabilization was determined using a FITC-conjugated anti-actin antibody. Cells were then washed twice in PB, resuspended in 500 μL of sheath fluid (BD PharMingen) to prevent clumping and analyzed by flow cytometry. For experiments in which purified cell populations were examined, cells were stained with an anti-CD3 or anti-CD19 antibody prior to permeabilization, and analyzed by gating on the CD3+ or CD19+ subsets.

### Example 5

### Western Blotting

Cells were pelleted, washed twice with PBS, and lysed for 30 min on ice in RIPA lysis buffer (50 mM Tris, pH 7.4, 150 mM NaCl 0.25% deoxycholate, 1% NP-40, and protease inhibitor cocktail, (Sigma)). Debris was removed by centrifugation for 15 min at 21,000 g at 4° C. Protein concentration was determined with the Bio-Rad Protein Assay reagent and equal amounts of protein (70-200 mg) were separated by SDS-PAGE electrophoresis on a 4-20% Tris-Glycine gel (Invitrogen) and transferred to Immobilon-P membrane (Millipore). The membranes were blocked with 5% non-fat dry milk/1×TBST (Tris-buffered saline with 0.1% Triton X-100) for 1 h at room temperature, hybridized with the appropriate antiserum diluted in 5% non-fat dry milk/1×TBST for 2 h at room temperature or overnight at 4° C., washed twice with 1×TBST, hybridized with the appropriate horseradish-peroxidase conjugated antibody diluted 1:5000 for 1 h at room temperature, and washed three times with 1×TBST. Hybridized bands were visualized using HyGlo chemiluminescent HRP antibody detection reagent (Denville Scientific) and exposed to film (Kodak). Antibodies used were anti-rat monoclonal to SFFV gp55 Env (7C10), diluted 1:100 detected with peroxidase labeled anti-rat secondary antibody (Amersham), goat anti-Rauscher p30 Gag, and NZB Xenotropic MuLV (provided by NCl), both diluted 1:2500 detected with peroxidase labeled anti-goat secondary antibody, (Santa Cruz Biotechnology).

### Example 6

### Viral Transmission

Frozen cell-free plasma and 0.22 μM filtered cell free supernatants from PBMC and T cell cultures were diluted 1:1 with tissue culture media and 600 μL were added to a six-well culture plate with the LNCaP cell line (50% confluent) or a million primary activated CD4+ T cells isolated from healthy donors. The plates were centrifuged 5 min at 1500 RPM, rotated 1800 and centrifuged again for 5 min. The entire cycle was repeated once and cells were diluted in their growth media.

For cell-cell transmission, 1×10⁶ T cells or PBMC without any IL-2 in the growth media were added to a six-well culture plate with the LNCaP cell line (50% confluent) in 1 mL of media for 3 h. After 1 hr, T cells in suspension were removed and the LNCaP cells were grown for several passages in the absence of IL-2 which caused any remaining T cells to die. At the times after transmission indicated, protein analysis was done by western blot and flow cytometry.

### Example 7

### Detection of XMRV in PBMCs and CFS subjects

This example illustrates prevalence of XMRV infection in subjects diagnosed with CFS, and XMRV infection of Peripheral Blood Mononuclear Cells (PBMCs). Methods are according to Examples 1-6 unless otherwise described.

Nucleic acids were screened from banked samples in a national tissue repository. For these experiments, nested PCR for the XMRV gag sequences were used as previously described (A. Urisman et al., PLoS Pathog 2, e25 (March, 2006)). PBMC samples were collected from a well-characterized CFS cohort between 2006 and 2008 and stored in TRIzol and later used for DNA and RNA isolation. Of the 101 CFS samples analyzed, 68 (67%) contained XMRV gag sequences as determined by nested PCR at the WPI (see e.g., FIG. 5).

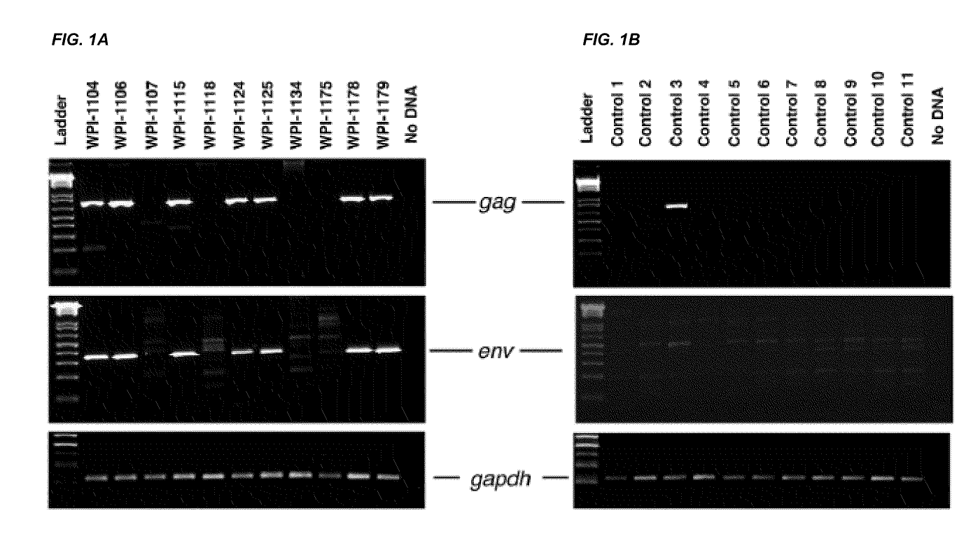

To verify the presence of XMRV in some of these subjects, segments of env (352 nt) and gag (736 nt) were PCR amplified and sequenced from PBMC DNA at the Cleveland Clinic from 7 of 11 CFS subjects (see.g., FIG. 1A). In all of the positive cases, the XMRV gag and env sequences were identical to those previously reported for XMRV (strains of XMRV VP62, VP35, and VP42) (see e.g., FIG. 6) (A. Urisman et al., PLoS Pathog 2, e25 (March, 2006)).

In the PCR analysis, 20 identical subject PBMC DNA specimens (stored at the NCl, Frederick, Md., since 2007) were analyzed. Analysis confirmed identical gag sequences in these subjects, thereby ruling out the possibility of lab contamination as a source.

In contrast, XMRV gag sequences were found in only 8 of 218 (3.7%) normal human PBMC DNA specimens from the same geographical (Western USA) region. A subset of these DNA samples were analyzed for both env and gag by PCR. In these analyses, only 1 of 11 was positive for gag and none for env (see e.g., FIG. 1B). These results were confirmed in three separate laboratories.

Further sequencing efforts produced full-length XMRV genomes from two CFS subjects and a partial genome from a third subject (see e.g., Table 2).

TABLE 2
Comparison of Nucleotide Sequences of XMRV Strains from Prostate
Cancer and Chronic Fatigue Syndrome Patients to XMRV VB62*

| | | | | Chronic Fatigue Syndrome Cases | | |
|---|---|---|---|---|---|---|
| | Prostate Cancer Cases | | | | | WPI-1104 |
| nt | VP 62 | VP 42 | VP 35 | WPI-1106 | WPI-1178C | (36-1152; |
| (number) | (4-8174 nt) | (1-8186 nt) | (1-8186 nt) | (36-8144 nt) | (36-8144 nt) | 5923-8147 nt) |
| 375 | | | A | | | |
| 450 | | | C | | | |
| 790 | | A | | | | |
| 1013 | | | T | | | |
| 1477 | | | G | | | |
| 1565 | | G | | | | |
| 1824 | | G | G | | | |
| 2413 | | | | A/G | | |
| 2416 | | | | | | |
| 2559 | | | A | | | |
| 2602 | | | A | | | |
| 2622 | | G | | | | |
| 4159 | | G | | | | |
| 4229 | | C deletion | | | | |
| 4236 | | G insertion | | | | |
| 4883 | | | | T | | |
| 4985 | | | | A | | |
| 5083 | | | T | | | |
| 5087 | | | A | | | |
| 5313 | | | | | | G |
| 5823 | | | | | C | |
| 5830 | | | | G | | |
| 6373 | | | | | G | |
| 6651 | | A | | | | |
| 7064 | | | G | | | |
| 7357 | | | A | | | |
| 7437 | | | | G | | |
| 7451 | | | | G | G | G |
| 7456 | | G | G | | G | |
| 7692 | | | | | T insertion | |
| 7782 | | G insertion | G insertion | | G insertion | |

*Accession numbers; VP62, EF185282; VP35, DQ241301; VP42, DQ241302

CFS XMRV strains WPI-1106 and WPI-1178 each contained six nucleotide differences from the reference strain XMRV VP62 (EF185282) from a prostate cancer case; however, with the exception of one nucleotide, these changes were at different locations in the XMRV genome. These results are consistent with independently acquired infections. In comparison, XMRV strains VP35 and VP42 from prostate cancer subjects had 13 and 10 nucleotide changes, respectively, with XMRV VP62.

These results confirm the presence of XMRV genomes in blood cells from CFS subjects and that XMRV sequences in these samples were highly similar (greater than 99%) to those found in prostate cancers.

### Example 8

### Presence of Infectious Virus in CFS Subjects

This example illustrates presence of infectious virus in subjects diagnosed with CFS. Methods are according to Examples 1-7 unless otherwise described.

To determine if infectious virus was present in PBMC from CFS subjects, an intracellular flow cytometry and western blot methods were developed using antibodies with novel viral specificities including monclonal antibodies to SFFV gp 55 Env (L. Wolff, R. Koller, S. Ruscetti, J Virol 43, 472 (August, 1982)), MuLV p30 Gag (B. Chesebro et al., Virology 127, 134 (May, 1983)), goat antisera to whole NZB xenotropic MuLV (Izui, S., et al., J. Exp. Med. 153, 1151-1160, 1981; Evans, L. H., et al., J. Virol. 64: 6176-6183, 1990), purified Rauscher MuLV gp70, p10, and p30 (Krakower, J. M., et al., J. Virol. 22: 331-339, 1977; Izui, S., et al., J. Exp. Med. 153, 1151-1160, 1981).

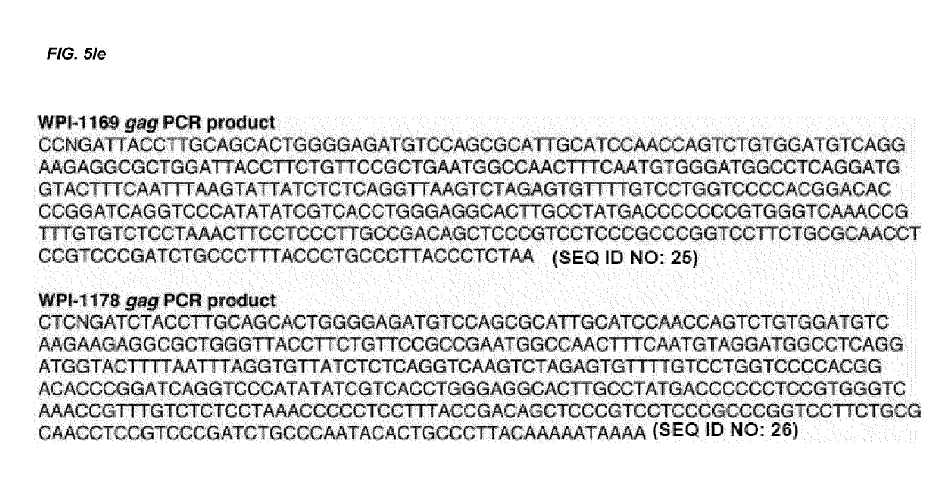

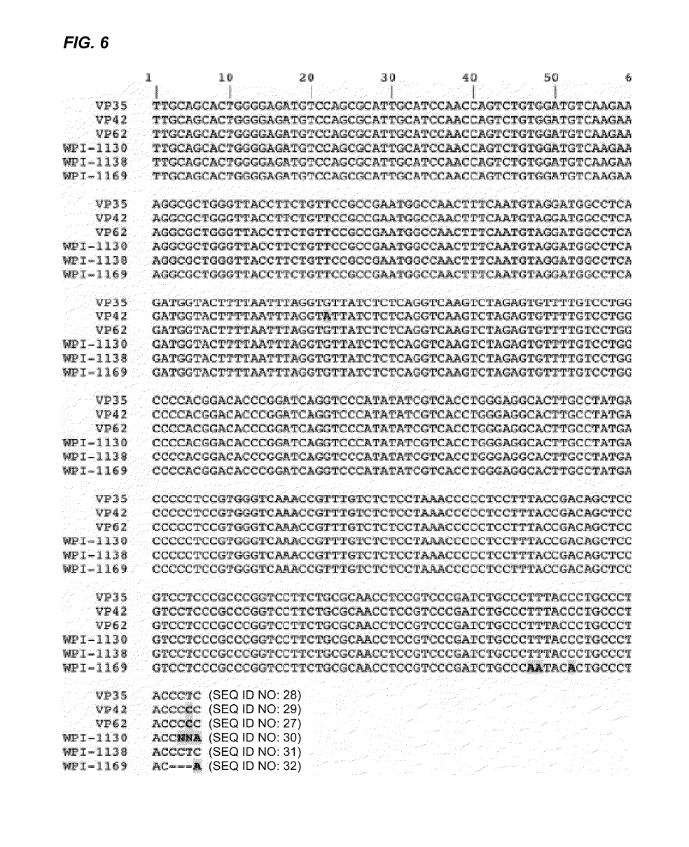

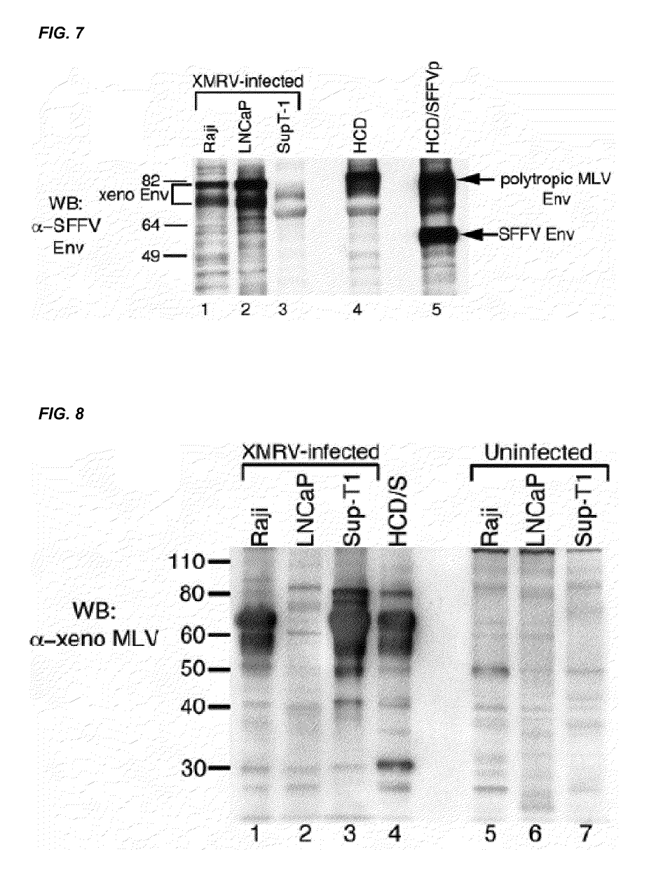

Results showed that the anti gp 55 Env, which recognizes the N-terminal regions of all tested mouse xenotropic and polytropic viruses, but not mouse ecotropic viruses, detected the human VP62 XMRV strain grown in Raji, LNCaP and Sup-T1 cells (see e.g., FIG. 7). The goat antisera against mouse xenotropic MuLV primarily recognized the Gag precursors in the human VP62 XMRV strain-infected, but not in uninfected cell lines (see e.g., FIG. 8).

Lymphocytes isolated from heparinized whole blood by ficoll density separation were activated using phytohemagglutinin (PHA) at 1 μg/ml in RPMI complete media and after 3 days, recombinant human interleukin 2 (rhlL-2) was added to the cultures at 20 units/ml (K. A Smith and F. W. Ruscetti, Adv Immunol. 31,137 (1981)).

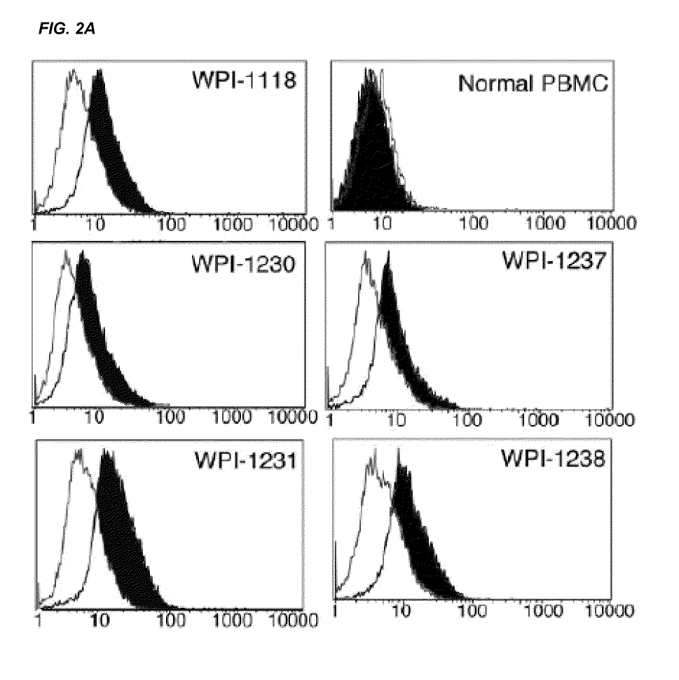

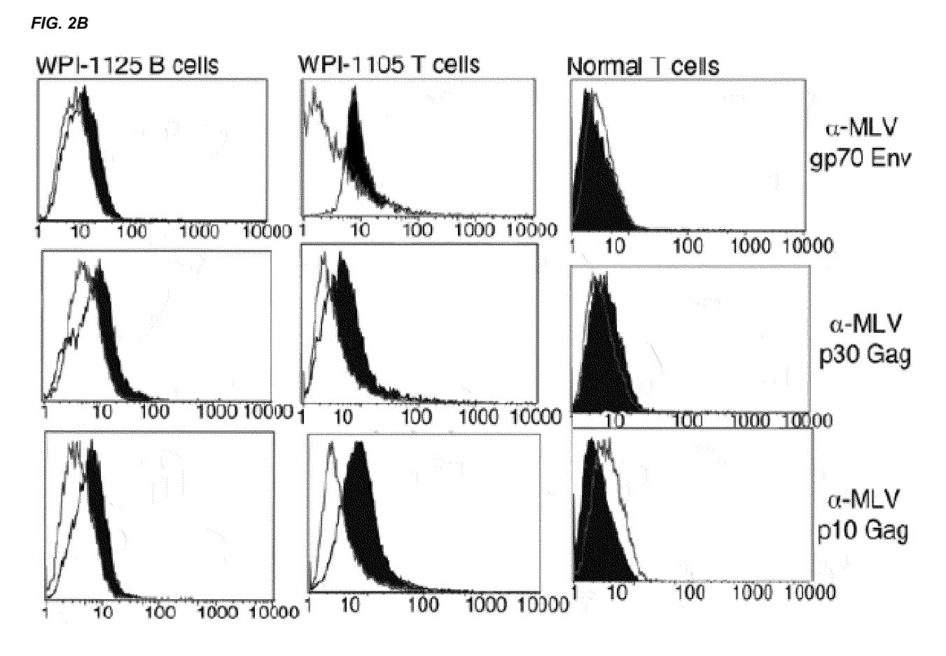

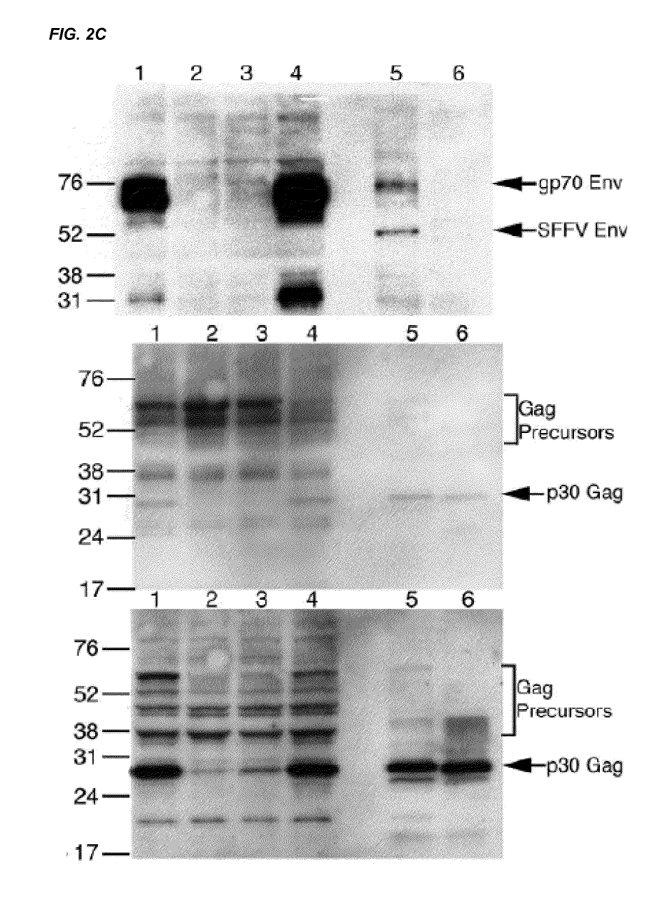

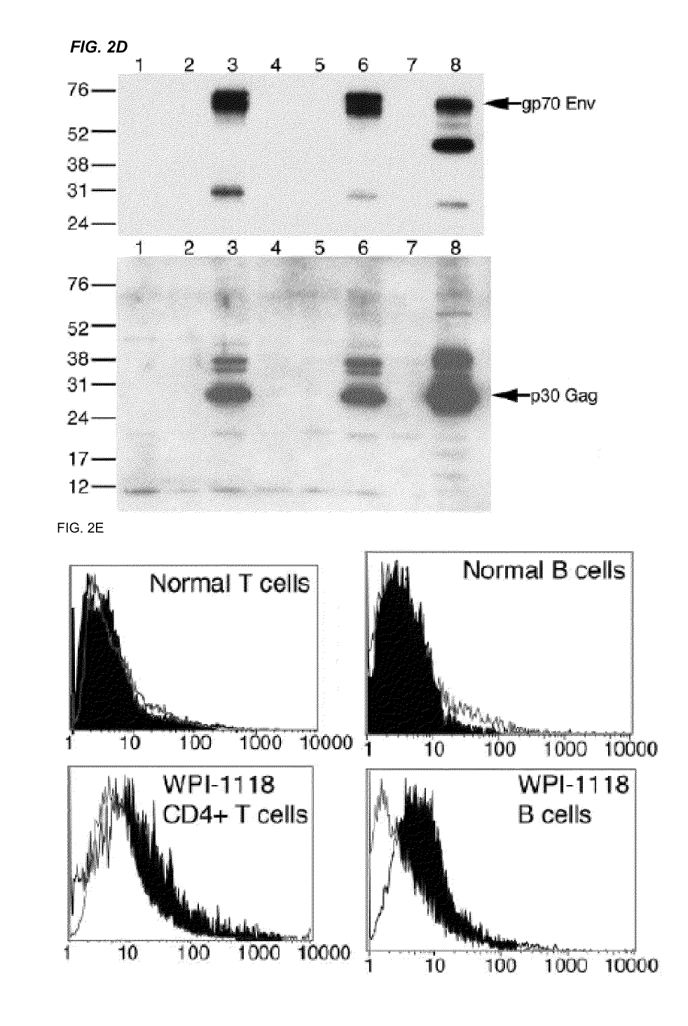

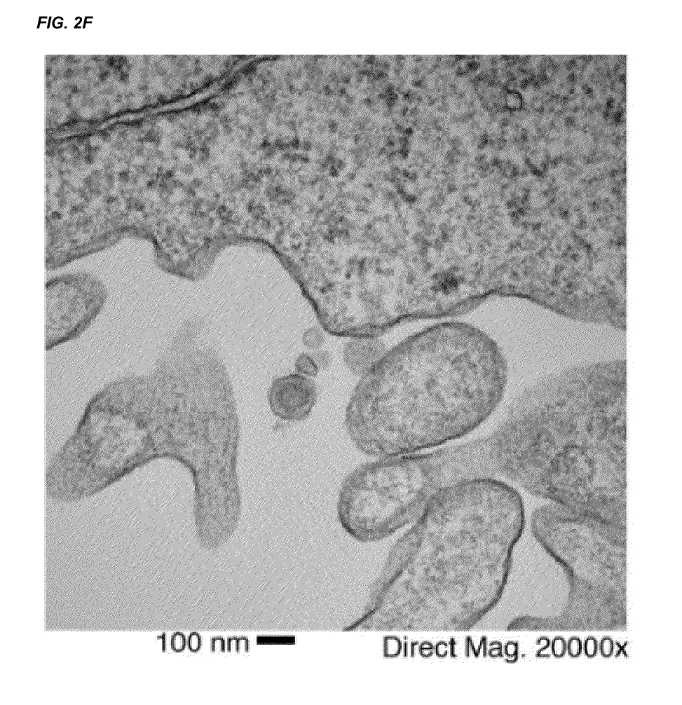

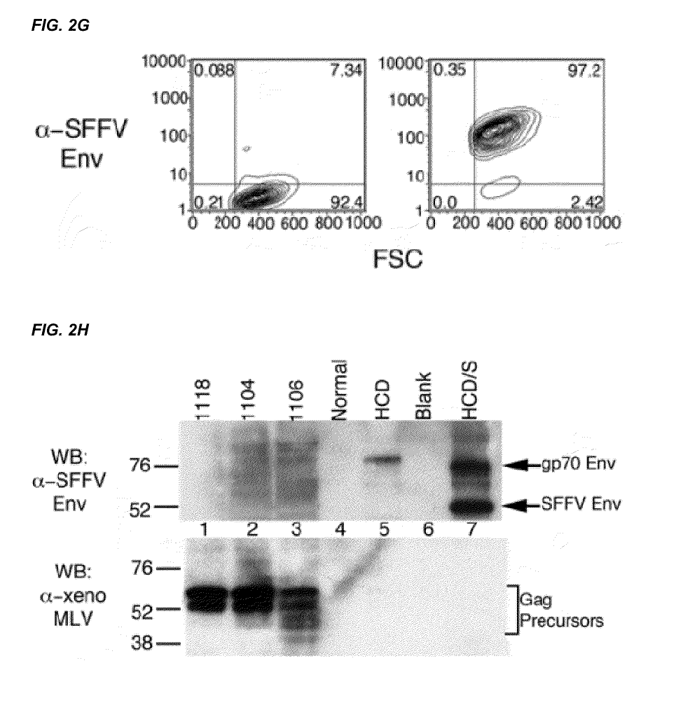

Results from intracellular staining by flow cytometry revealed 19 out of 30 subject PBMC samples tested positive for at least one viral protein (p30) (see e.g., FIG. 2A) using a monoclonal antibody to MuLV p30 and several for multiple MuLV proteins (see e.g., FIG. 2B) using polyclonal antisera to purified viral proteins, while 16 healthy control PBMC cultures tested negative (see e.g., FIG. 2A, B). These results were also confirmed by western analysis (see e.g., FIG. 2C) using antibodies to SFFV gp55 Env, mouse xenotropic virus and Rauscher p30 Gag. Variations in XMRV protein expression in PBMC were observed in five CFS subjects where no XMRV expression was seen in PBMC from five normal donors (see e.g., FIG. 2D).

To determine the cell type tropism of XMRV, B and T cells from one subject's PBMC were isolated by immunomagnetic bead separation and analyzed using a monoclonal antibody to MuLV.

Results demonstrated that both activated T and B cells were infected with XMRV p30 Gag (see e.g., FIG. 2E). Furthermore, greater than 95% of the cells in a B-cell line developed from one subject in 2007 were positive for XMRV Env using a monoclonal antibody to SFFV gp 55 Env (see e.g., FIG. 2G). XMRV protein expression in CFS subject-derived activated T and B cells grown for 42 days in culture was confirmed by Western blots using antibodies to SFFV Env and xenotropic MLV (see e.g., FIG. 2H).

Transmission electron microscopy of an unactivated T-cell culture revealed a 90-100 nM diameter particle (see e.g., FIG. 2F) consistent with a type C retrovirus (B. J. Poiesz et al., Proc Natl Acad Sci USA 77, 7415 (December, 1980)). XRMV expression in unactivated T and B cells was also confirmed by western analysis (see e.g., FIG. 2H) using antibodies to SFFV gp55 Env, mouse xenotropic virus and Raucher p30 Gag.

These results demonstrate that XMRV can infect PBMCs, such as B cells and T cells.

### Example 9

### Transmission of XMRV

This example illustrates transmission of XMRV. Methods are according to Examples 1-8 unless otherwise described.

PBMC activated with PHA and IL-2 (B. J. Poiesz et al., Proc Natl Acad Sci USA 77, 6815 (November, 1980); J. A. Mikovits, N. C. Lohrey, R. Schulof, J. Courtless, F. W. Ruscetti, J Clin Invest 90, 1486 (October, 1992)) were cultured to generate infectious XMRV, which was transmitted to an XMRV indicator cell line, LNCaP. LNCaP is a metastatic androgen responsive prostate cancer cell line with defects in both the JAK-STAT pathway and the RNase L pathway (G. P. Dunn, K. C. Sheehan, L. J. Old, R. D. Schreiber, Cancer Res 65, 3447 (Apr. 15, 2005), Y. Xiang et al., Cancer Res 63, 6795 (Oct. 15, 2003)). These cells were previously shown to be permissive for XMRV infection (B. Dong et al., Proc Natl Acad Sci USA 104, 1655 (Jan. 30, 2007)).

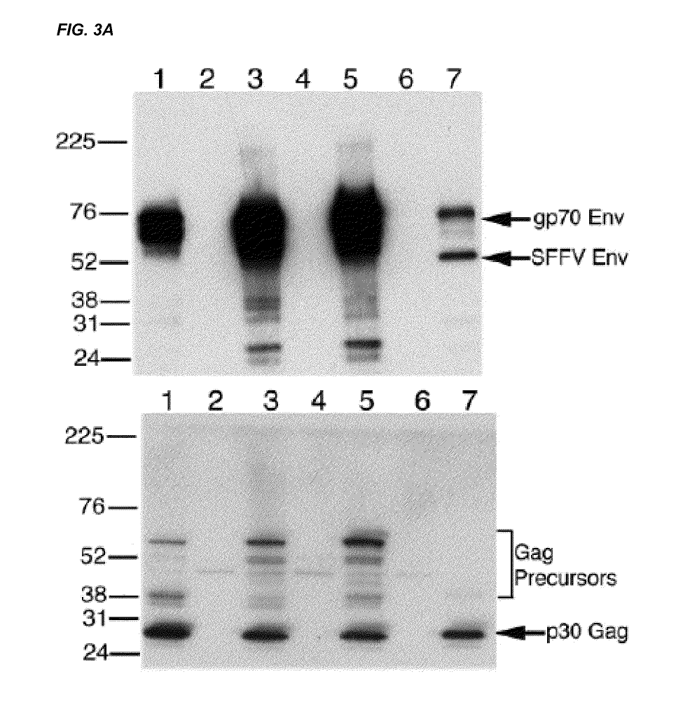

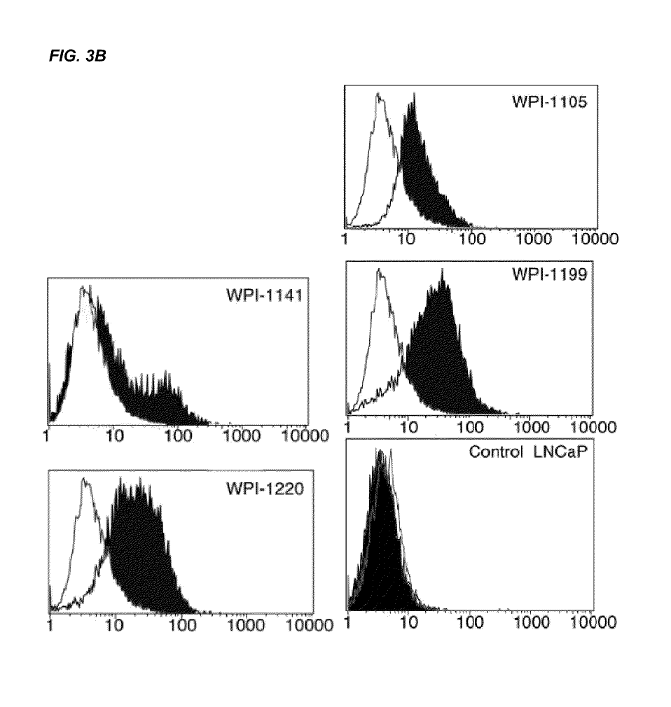

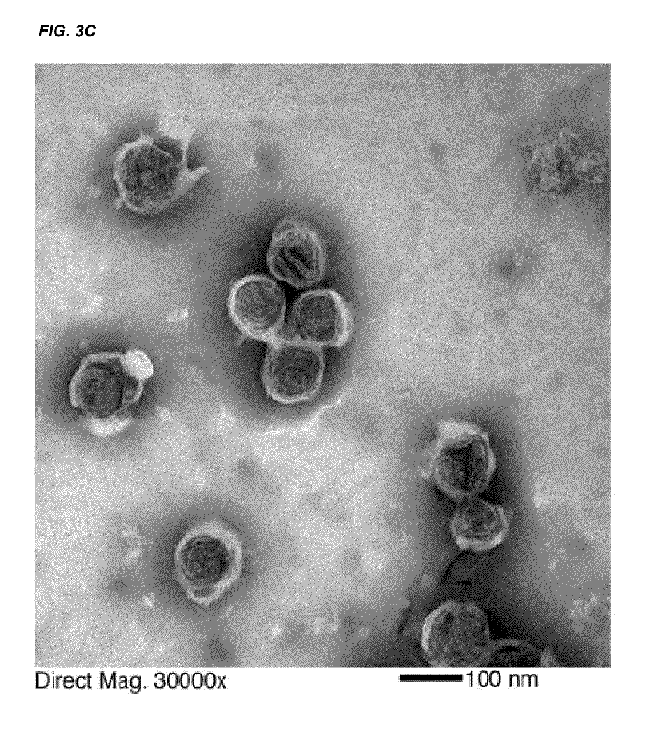

Lymphocytes from subjects diagnosed with CFS, following activation and co-culture with LNCaP, expressed XMRV Env and multiple XRMV Gag proteins as shown by western blot analysis (see e.g., FIG. 3A). Furthermore, viral p30 Gag expression was observed in these LNCaP cells using intracellular flow cytometry (see e.g., FIG. 3B).

In some experiments, cell free supernatants from the co-cultures were filtered through 0.22 μM filter to remove cells and debris. Virus was concentrated by ultracentrifugation over a 20% glycerol cushion, producing abundant 90-100 nM diameter particles consistent with a type C retrovirus (see e.g., FIG. 3C) (B. J. Poiesz et al., Proc Natl Acad Sci USA 77, 7415 (December, 1980)).

### Example 10

### Cell-Associated and Cell-Free Transmission of XMRV

This example illustrates cell-associated and cell-free transmission of XMRV. Methods are according to Examples 1-9 unless otherwise described.

To investigate possible routes of transmission, plasma from heparinized blood that had been frozen in liquid nitrogen within one hour of blood draw and stored on dry ice was examined. To determine if infectious XMRV could be detected in plasma, a virus isolation spinning protocol was employed, which has previously been shown to greatly enhance the ability of retroviruses and Kaposi's sarcoma-associated herpes virus, to infect different cell types in vitro (G. R. Pietroboni, G. B. Harnett, M. R. Bucens, J Virol Methods 24, 85 (April-May, 1989); S. M. Yoo et al., J Virol Methods (Sep. 19, 2008)). After co-culture with plasma, the LNCaP cells were sub-cultured for 2-4 passages.

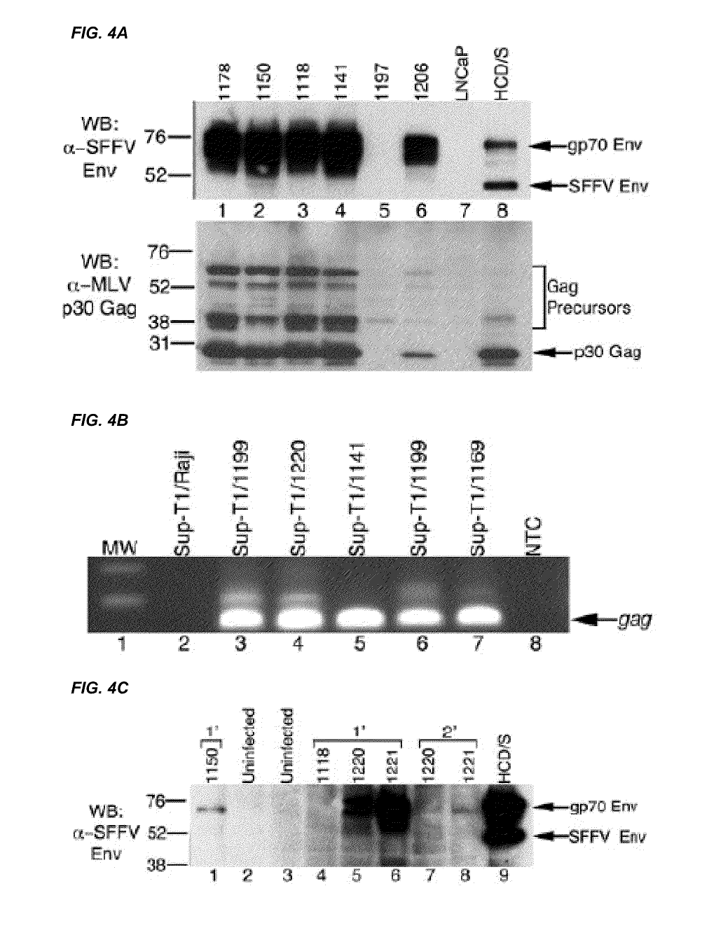

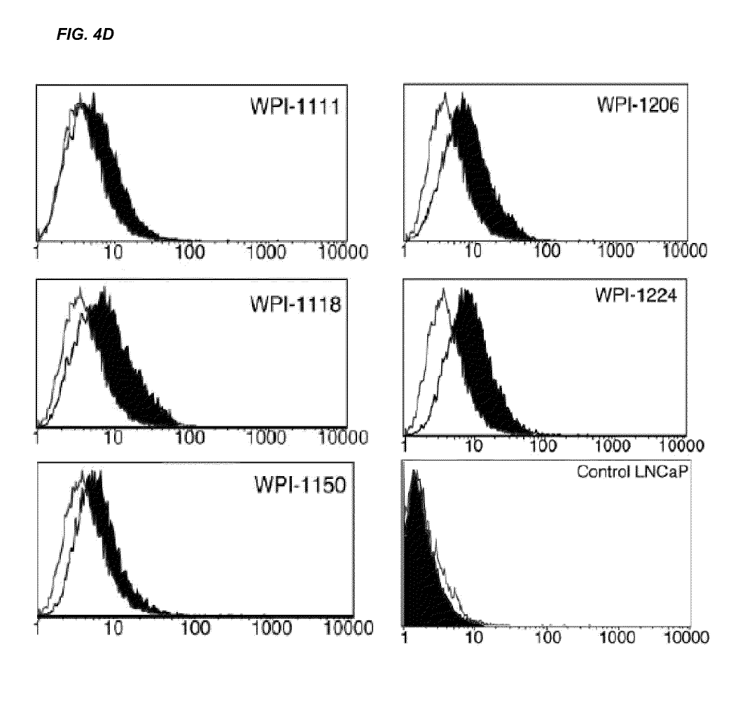

Results showed that both XMRV gp70 Env and p30 Gag were abundantly expressed in LNCaP cells incubated with plasma samples from 10 out of 12 plasmas, whereas no viral protein expression was detected in LNCaP cells incubated with plasma samples from 12 healthy donors (see e.g., FIG. 4A). Likewise, LNCaP cells incubated with subject plasma tested positive for XMRV p30 Gag in IFC assays (see e.g., FIG. 4B).

Results also showed cell-free transmission of XMRV from the PBMCs of CFS subjects to the Tcell line SupT1 (FIG. 4B). Results also showed both primary and secondary transmission of cell-free virus from the activated T cells of CFS subjects to normal T cell cultures (FIG. 4C) and from plasma of CFS subjects to human foreskin fibroblast cells (FIG. 4D).

These results indicate both cell-associated and cell-free routes of transmission of XMRV in a human population.

### Example 11

### Flow Cytometry for Detection of Antiviral Antibodies in CFS Plasma

This example demonstrates flow cytometry for detection of antiviral antibodies in CFS plasma. Methods are according to Examples 1-10 unless otherwise described.

Murine cell lines BaF3ER and BAF3ER-SFFV Env (Nishigaki, K., et al., J. Virol. 75: 7893-7903, 2001) comprising gp55 env plasmid to express SFFV gp 55 env were grown in 2 units/ml of Epo in RPMI 1640 in 7% FCS. 500,000 cells per sample in log phase were used as targets for direct staining. Cell lines were first washed in wash buffer (2% FBS, 0.02% Na Azide, PBS) and resuspended in 200 μl of BSA staining buffer (BD PharMingen, San Jose, Calif.). Subject plasma was thawed rapidly and used at 20 μl or 2 μl per tube (1:10 and 1:100 respectively). Samples were incubated at 4° C. or on ice for 30 minutes. Cells were then washed with 0.5 mL of the wash buffer. Tubes were centrifuged at 800 rpm for 5 minutes, the supernatant was removed and samples were blotted on a towel. Next, 100 μL of the following working solution was added: 5 μL human A/B sera, 1 μL biotin-labeled anti-human IgG (for human plasma or biotin-labeled anti rat IgG (for 7C10 monoclonal antibody against SFFV Env, Wolff, L. et al., J. Virol. 3: 72-81 (1982)) (Ebioscience, San Diego, Calif.), 1 μL of strep/avidin phycoerythrin (PE), 94 μL cold staining buffer. Samples were then incubated at 4° C. for 20 minutes, washed with 0.5 mL of the wash buffer, and spun at 800 rpm for 5 minutes before being analyzed by flow cytometry. For the competition experiments, 100 μL of cold staining buffer and 10 μL of human plasma were added to each tube prior to addition of either anti-SFFV Env mAb (7c10) or Y3 myeloma supernatant (control). Samples were incubated at 4° C. or on ice for 20 minutes, washed with 0.5 mL of wash buffer and spun at 800 rpm for 5 minutes before being analyzed by flow cytometry.

Subject Samples.

Banked samples were selected from subjects fulfilling the 1994 CDC Fukuda Criteria for Chronic Fatigue Syndrome (Fukuda, K., et al., Ann. Intern. Med. 121: 953-959, 1994) and the 2003 Canadian Consensus Criteria (CCC) for Chronic Fatigue Syndrome/myalgic encephalomyelitis (CFS/ME) and presenting with severe disability. Samples were selected from several regions of the United States where outbreaks of CFS had been documented (DeFreitas, E., et al., Proc. Nat'l. Acad. Sci. USA 88: 2922-2926, 1991). These are subjects that have been seen in private medical practices, and their diagnosis of CFS is based upon prolonged disabling fatigue and the presence of cognitive deficits and reproducible immunological abnormalities. These included but were not limited to perturbations of the 2-5A synthetase/RNase L antiviral pathway, low natural killer cell cytotoxicity (as measured by standard diagnostic assays), and elevated cytokines, particularly interleukin-6 and interleukin-8. In addition to these immunological abnormalities, the subjects characteristically demonstrated impaired exercise performance with extremely low VO₂ max measured on stress testing. The subjects had been seen over a prolonged period of time and multiple longitudinal observations of the clinical and laboratory abnormalities had been documented.

### Example 12

### Direct Detection of Antibody Against XMRV in Plasma from CFS Subjects

This example demonstrates direct detection of antibody against XMRV in plasma from CFS subjects. Methods are according to Examples 1-11 unless otherwise described.

The above described demonstration that infectious virus is present in both T and B lymphocytes from CFS subjects is consistent with the tropism of other well-documented targets of human retroviral infection (B. J. Poiesz et al., Proc Natl Acad Sci USA 77, 7415 1980; J. C. Chermann et al., Antibiot Chemother 32, 48, 1983). It was further investigated whether XMRV stimulated an immune response in these subjects.

A flow cytometry assay was developed for detecting antibodies to XMRV ENV by exploiting its close homology to SFFV Env. This assay uses a murine pro B cell line, BAF-3 (control) and BAF-3 stably expressing SFFVgp55 ENV. In these experiments, plasma from CFS subjects or normal healthy controls was diluted 1:10, reacted with BaF3-ER or BaF3ERSFFV Env cells and analyzed by intracellular flow cytometry (IFC).

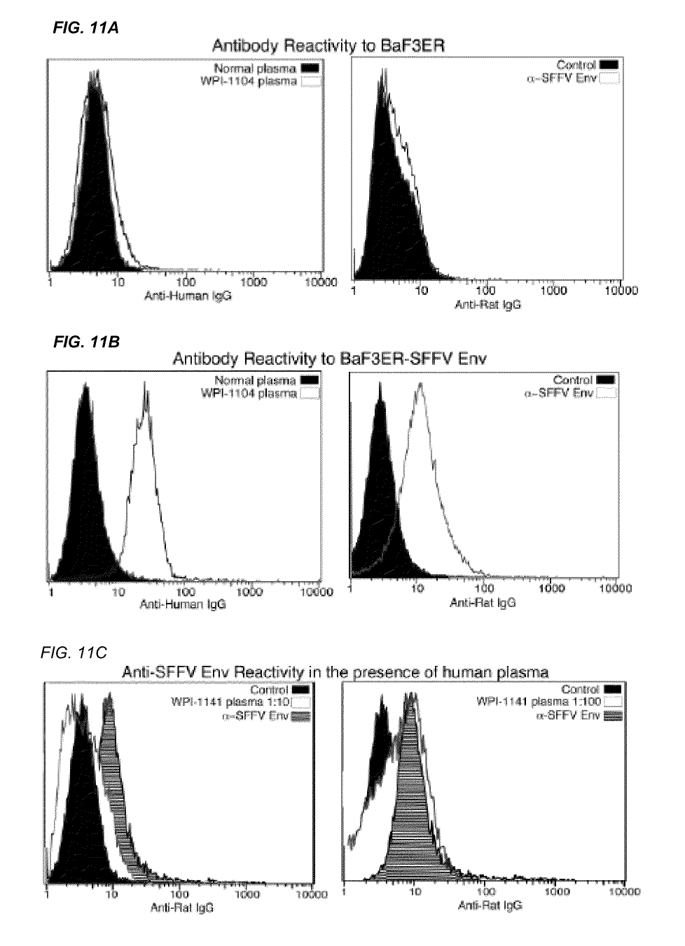

Plasma from 9 out of 18 CFS subjects infected with XMRV reacted with a mouse B cell line expressing recombinant SFFV Env (BaF3ERSFFV-Env) (see e.g., FIG. 11B) but not to SFFV Env negative control cells (BaF3ER) (see e.g., FIG. 11A), analogous to the binding of the SFFV Env mAb to these cells. In contrast, plasma from seven healthy donors did not react (see e.g., FIG. 11). Furthermore, all nine positive plasma samples from CFS subjects but none of the plasma samples from healthy donors blocked the binding of the SFFV Env mAb to SFFV Env on the cell surface.

These results support that CFS subjects mount a specific immune response to XMRV.

### Example 13

### Detection of Antibody Against XMRV in Plasma from CFS Subjects

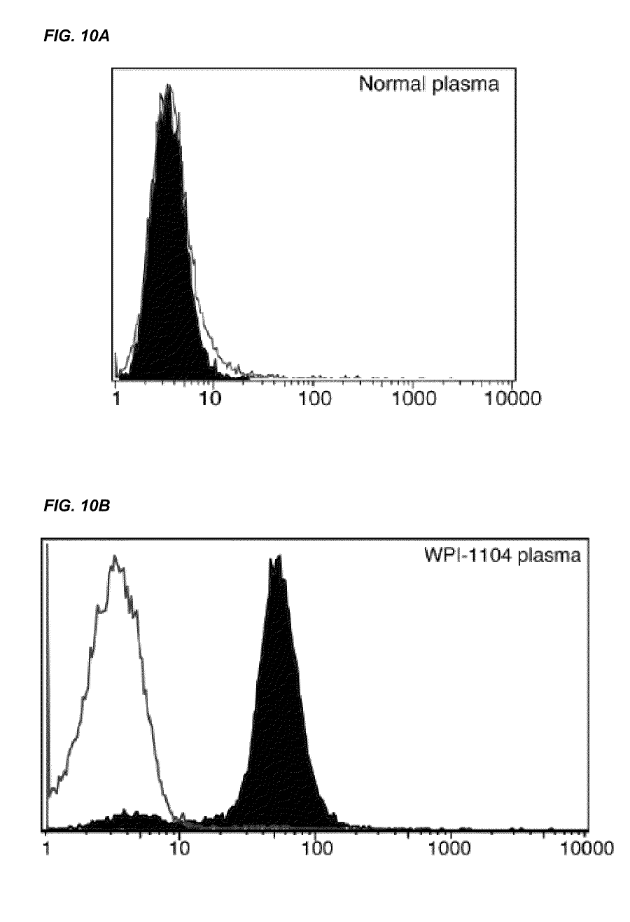

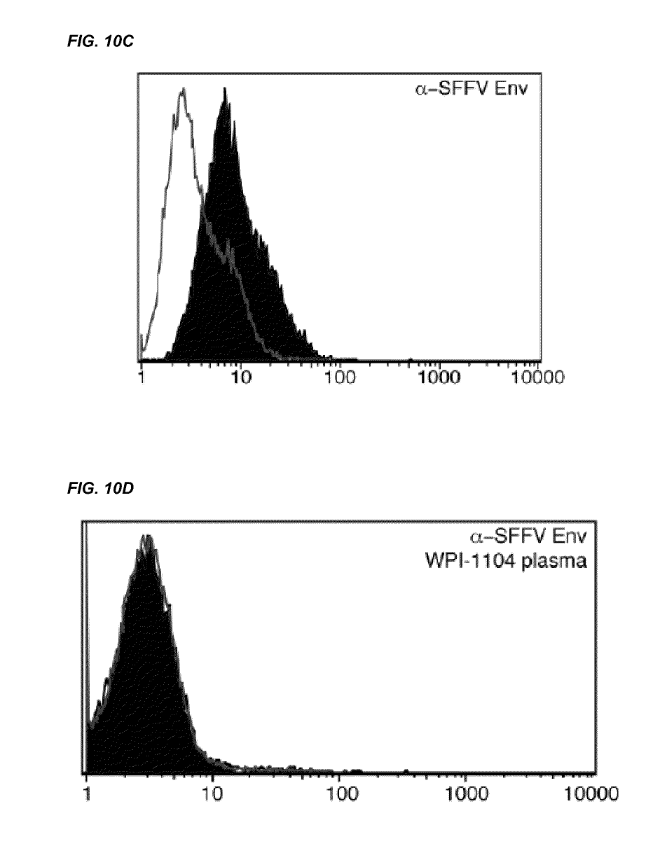

This example illustrates detection of antibody against XMRV in plasma from CFS subjects. In these experiments, samples of human plasma were assayed by flow cytometry for presence of antibody against XMRV using direct and competitive assays (see e.g., FIG. 10). Methods are according to Examples 1-12 unless otherwise described.

Direct assay of binding of normal plasma with BaF-3-SFFV ENV was negative (see e.g, FIG. 10A). Furthermore, direct assay of binding of normal plasma with BaF-3 ER FC was also negative (see e.g., FIG. 10B). But a plasma sample from a subject who was previously diagnosed clinically with CFS (designated subject 1104) was found to comprise antibody against XMRV (see e.g., FIG. 10B, black area, a 1:10 dilution of plasma was positive in this assay). FIG. 10C illustrates direct binding of 2 μL 7C10 monoclonal anti SFFV Env antibody to BAF3-SFFV gp55 cell line (black area) vs. binding to BaF-3 ER control (light area). FIG. 10D illustrates competition for binding of 7C10 to BAF3-SFFV gp55 cell line by 1:10 dilution of plasma from subject 1104.

These data show specificity of antibody in CFS subject 1104 plasma, and demonstrate the ability of human sera to block 7C10 binding (black area totally overlaps light negative area).

### Example 14

### Detection of Antibodies Against XMRV in Sera of Subjects Using Direct and Competitive Assays

This example demonstrates detection of antibodies against XMRV in sera of subjects using direct and competitive assays (see e.g., FIG. 11). Methods are according to Examples 1-13 unless otherwise described.

It was investigated whether XMRV stimulates an immune response in CFS subjects. For this purpose, a flow cytometry assay was developed that allowed detection of antibodies to XMRV Env by exploiting its close homology to SFFV Env (Wolff, L., et al., Proc. Nat'l. Acad. Sci. USA 80: 4718-4722, 1983).

Results showed no direct binding on BAF3ER control cells (see e.g., FIG. 11A). Left panel: binding of human plasma at 1:10 dilution as detected by anti human IgG; right panel: no binding of anti-SFFV env monoclonal (7C10) at 1:10 dilution as detected using an anti rat IgG. Y3 rat hybridoma supernatant served as control.

Results showed direct binding on BAF3ER-SFFV Env cells (see e.g., FIG. 11B). Left panel illustrates direct binding of human CFS from subject 1104 but not normal plasma at 1:10 dilution as detected by anti human IgG; right panel illustrates direct binding of anti-SFFV Env monoclonal at 1:10 as detected by anti rat IgG.

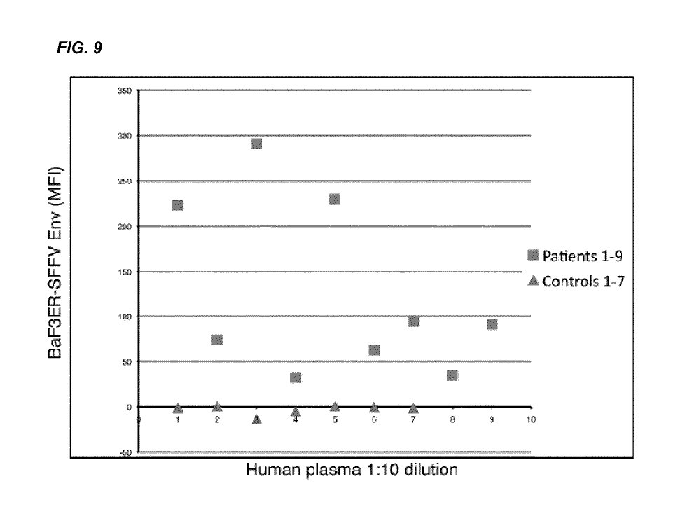

In these experiments, plasma from 9 out of 18 CFS subjects infected with XMRV reacted with a mouse B cell line expressing recombinant SFFV Env (BaF3ER-SFFV-Env) (see e.g., FIG. 11B) but not to SFFV Env negative control cells (BaF3ER) (see e.g., FIG. 11A), analogous to the binding of the SFFV Env mAb to these cells (see e.g., FIG. 11A-B, FIG. 9). In contrast, plasma from seven healthy donors did not react (see e.g., FIG. 11A, FIG. 9).

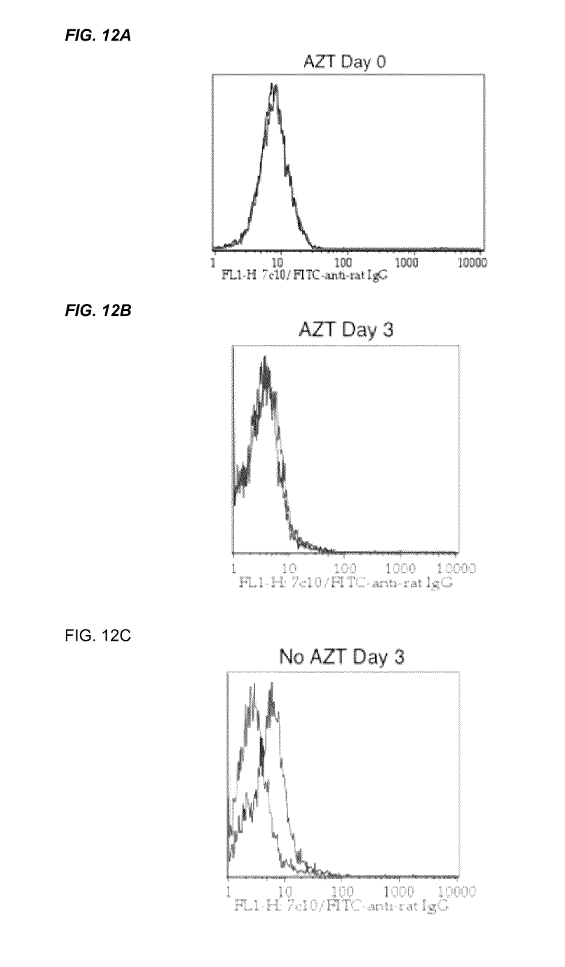

Further experiments indicate that plasma from a CFS subject can block binding of a rat anti-SFFV Env mAb to BaF3ER-SFFV Env cells. In these experiments, all nine positive plasma samples from CFS subjects, but none of the plasma samples from healthy donors blocked the binding of the SFFV Env mAb to SFFV Env on the cell surface. CFS plasma competes with anti-SFFV Env for binding to BAF3ER-SFFV Env cells (see e.g., FIG. 12C). Left panel: CFS plasma from subject 1141, diluted 1:10 (white area) eliminates most of the anti-SFFV Env binding (striped area) and overlaps with the negative control (black area). Right panel: CFS plasma diluted 1:100 (white area) eliminates less of the anti-SFFV Env binding (striped area) and overlaps much more with the positive than the negative control (black area). It was found that at dilutions of 1:10 and 1:100, plasma from several of the CFS subjects', but not normal plasma, significantly blocked anti-SFFV antibody binding.

These experiments show that plasma from a CFS subject can compete with an anti-SFFV Env mAb for binding to cells comprising SFFV Env.

These data indicate that CFS subjects mount a specific immune response to XMRV. Furthermore, these results demonstrate that an antibody against XMRV can be reliably detected in infected individuals using the disclosed methods, including individuals having an XMRV-related disease such as CFS.

### Example 15

### RNA and DNA Isolation

This example demonstrates nucleic acid isolation. Methods are according to Examples 1-14 unless otherwise described.

TRIzol method was used to extract RNA as follows. 2-5 million PBMC or 250 μL plasma were added to 1 mL TRIzol reagent or 750 uL TRIzol LS reagent and RNA was prepared according to manufacturer's instructions. The final RNA pellet was suspended in 14 to 30 μL of RNase-free water, and quantified using Quant-iT RiboGreen RNA (Invitrogen).

Reverse transcription (RT) was performed with the SuperScript VILO cDNA synthesis kit (Invitrogen). cDNA was made from a mixture of oligo d(T) and random primer using 2.5 μg total RNA. Subsequent optimization of nested PCR revealed that an increase in starting template leads to a reduction in false negatives. Identification of XMRV gag and env genes was performed by PCR in separate reactions.

Alternatively, cDNA was synthesized followed immediately by the first round of PCR amplification using SuperScript One-Step RT-PCR with a Taq enzyme according to the manufacturer's instructions, using 0.5 μg RNA as template and the previously described GAG-O-F (F542) and GAG-O-R (R1153) as the sense and antisense primers, respectively (Urisman et al., PLOS Pathology, 2006, 2:e25-31, unless otherwise specified). The second round of PCR was performed using 5 μL of the reaction mix, the primers GAG-I-F (F603) and GAG-I-R (R1015) (Urisman et al., PLOS Pathology, 2006, 2:e25-31, unless otherwise specificed) using 0.5 μl Taq polymerase and Invitrogen PCR buffer, supplemented with an additional 0.5 mM MgCl₂. The PCR products were then separated on a 1% agarose gel, and the PCR products of the expected size (413 bp) were recovered with a QIAEX II gel extraction kit (Qiagen) and sequenced.

### Example 16

### Real Time PCR for Detection of Mouse Contamination

A highly sensitive assay containing two Taqman probes was developed to detect the presence of mouse contamination. Methods are according to Examples 1-15 unless otherwise described.

A 50 μL reaction volume was formed with: 500 ng to 1000 ng DNA/cDNA, 5 μL of 10× AmpliTaq buffer, 1 μL of 10 mM dNTPs, 3 μL of 25 mM MgCl2, 0.5 μL AmpliTaq Gold Taq, 4 μL of each of 4 μM forward and reverse oligonucleotide primers (below), 4 μL of each 2 μM oligonucleotide probe.

For identification of murine mitochondrial cytochrome oxidase, MCox2-F2 (5′-TTCTACCAGCTGTAATCCTTA-3′) (SEQ ID NO: 21) and MCox2-R1 (5′-GTTTTAGGTCGTTTGTTGGGAT-3′) (SEQ ID NO: 22) were used as forward and reverse primers. For probes, MCox2-PR1 (5′-FAM-CGTAGCTTCAGTATCATT GGTGCCC TATGGT-BHQ-3′) (SEQ ID NO: 23) and MCox2-P1 (5′-FAM-TTGCTCTCCCCTCTCTACGCATTCTA-BHQ-3′) (SEQ ID NO: 24) were used.

Cycling conditions were as follows: 95° C. for 9 min initial denaturation was performed for every reaction followed by a cycle of 95° C. for 30 seconds, and a cycle of 62° C. for 30 seconds; this cycle was repeated 55 times. A cloned PCR product amplified from mouse RAW cells using primers MCox2-F2 and MCox-R1 was used as a positive control.

### Example 17

### Viral Amplification by Transmission to LNCaP

This method demonstrates viral amplification by transmission to LNCaP. Methods are according to Examples 1-16 unless otherwise described.

Plasma from 20 mL of anticoagulant blood was flash frozen and PBMC isolated by ficoll-hypaque density centrifugation. The PBMC were activated for three days in RPMI complete media supplemented with PHA (1 μg/mL) and IL-2 (20 units/mL). In 15 mL centrifuge tubes, 5×10⁵ detached LNCaP, 1×10⁵ activated PBMC free of IL-2, and 50 μL of autologous plasma in 250 μL of RPMI complete media were centrifuged for 10 min at 1500 rpm. The entire contents of the cell pellet were cultured in a T-25 flask in complete RPMI 1640 media for 4-5 days until the LNCaP cells were confluent. DNA was then extracted from the cells and nested PCR for gag was performed as previously described. A companion negative normal donor was run under the same conditions.

### Example 18

### Flow Cytometry for XMRV Proteins in PBMC

This example demonstrates flow cytometry for XMRV proteins in PBMC. Methods are according to Examples 1-17 unless otherwise described.

PBMC were isolated by ficoll-hypaque centrifugation followed by two washes, and then activated for three or six days with PHA and IL-2 in the presence and absence of 50 nM AZT. Then suspension cells (either unactivated at day 0 or activated) were incubated for 15 min at room temperature in 1 mL of paraformaldehyde (4% w/v in PBS), washed in permeabilization wash buffer (0.5% saponin, 0.1% sodium azide, 2% human AB sera in PBS) (PWB), and resuspended in 300 μL of permeabilization buffer (PBS with 2.5% saponin) (PB).

After incubating at 22° C. for 20 minutes, 5 μL of human AB sera and either a rat anti-MuLV p30 or rat anti-SFFV gp55 Env, goat anti Rauscher gp 70 Env, p30 Gag, and p10 Gag or the appropriate isotype control (anti-rat IgG, rat hybridoma supernatant, or preimmune goat serum) were added, and the cells were incubated at 4° C. for an additional 30 minutes. Cells were then washed in PWB, resuspended in 100 μL of PB with 3 μL (0.6 μg) of FITC-conjugated goat anti-rat IgG or rabbit anti-goat antibody, and incubated for 20 minutes at 4° C.

The efficiency of permeabilization was determined using a FITC-conjugated anti-actin antibody. Cells were then washed twice in PB, resuspended in 500 μL of sheath fluid (BD PharMingen) to prevent clumping, and analyzed by flow cytometry.

### Example 19

### Detection of XMRV by Co-Culture on LNCaP Versus Whole Blood DNA PCR

This example demonstrates detection of XMRV by co-culture on LNCaP versus whole blood DNA PCR. Methods are according to Examples 1-18 unless otherwise described.

Fifty-one CFS subject samples were tested for XMRV according to either co-culture on LNCaP or whole blood DNA PCR. Results showed 37/51 positive by nested PCR following co-culture with LNCaP but only 11/51 positive following nested PCR from DNA from unfractionated PBMC.

### Example 20

### Detection of XMRV in Activated and Unactivated PBMC

This example shows XMRV is detected in activated PBMC but not in unactivated PBMC. Methods are according to Examples 1-19 unless otherwise described.

PBMC were separated from blood samples of CFS subjects. Separated PBMC were tested by intracellular flow cytometry, unactivated at time 0, PHA/IL-2 activated in the presence and absence of 50 nM azidothymidine (AZT) for three and six days (Sakuma et al., Virology 2009. 397; 1-6).

Results showed that no subject's PBMC were positive for XMRV Env or p30 Gag at time zero; by day three, 3 of 7 were positive and by day six, 6 of 7 were positive. None of the AZT treated PBMC cultures (all of which were greater than 90% viable after six days) were found positive (see e.g., FIG. 12).

These results indicate that XMRV positive activated PBMC are a result of viral infection from cell-associated virions or proviral copy numbers below the limit of detection by PCR and that XMRV spreads throughout the culture during proliferation suggesting that PBMC may not be the main reservoir for XMRV.

### Example 21

### Phylogenetic Analysis

To exclude the possibility of detecting a murine leukemia virus (MLV) laboratory contaminant, the phylogenetic relationship among endogenous (non-ecotropic) MLV sequences, XMRV sequences, and sequences from CFS subjects 1104, 1106, and 1178 was determined. Methods are according to Examples 1-20 unless otherwise described.

Sequences were aligned using ClustalX (S3). Clustal alignments were imported into MEGA4 to generate neighbor-joining trees using the Kimura 2-parameter plus ┌ distribution (K80+┌) distance model (S4). Free parameters were reduced to the K80 model, and α values were estimated from the data set using a maximum likelihood approach in PAUP*4.0 (Sinauer Associates, Inc. Publishers, Sunderland, Mass., USA). The bootstrap consensus tree inferred from 1000 replicates is taken to represent the evolutionary history of the taxa analyzed. Accession numbers were acquired from GenBank: FLV (NC_{—}001940), MoMLV (NC_{—}001501), XMRV VP35 (DQ241301), XMRV VP42 (DQ241302), XMRV VP62 (EF185282). Genomic Nonecotropic MLV Provirus Sequences were downloaded from PLOS Genetics.

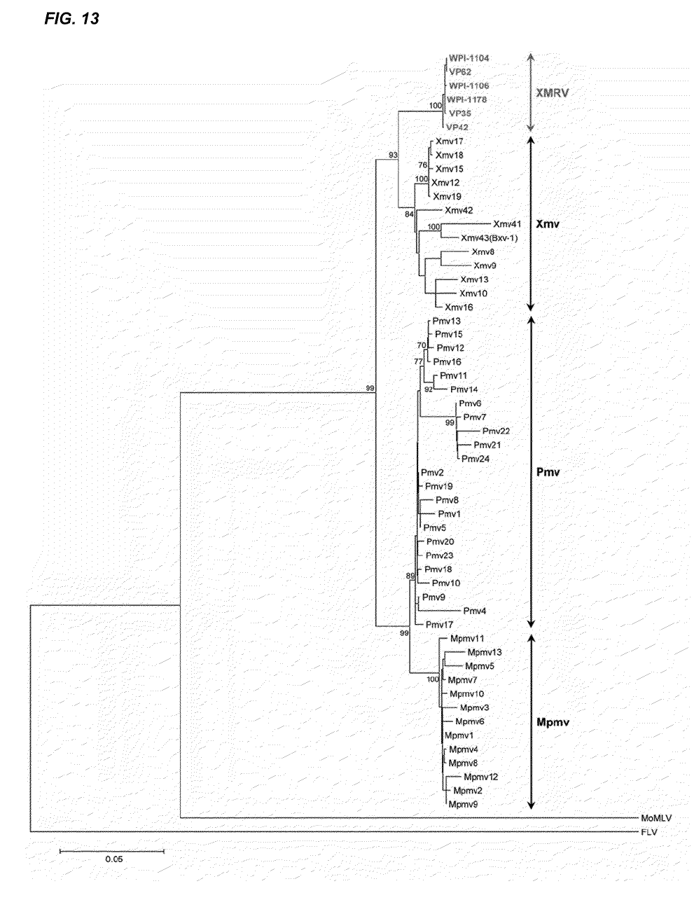

Results showed that XMRV sequences from the CFS subjects clustered with the XMRV sequences from prostate cancer cases and formed a branch distinct from non-ecotropic MLVs common in inbred mouse strains (see e.g., FIG. 13).

Thus, the virus detected in the CFS subjects' blood samples is unlikely to be a contaminant.

### Example 22

### XMRV Detection in Subjects with Chronic Lyme Disease

This example demonstrates the presence of XMRV in subjects having chronic lyme disease. Methods are according to Examples 1-21 unless otherwise described.

Samples from twenty subjects diagnosed with chronic lyme disease were tested for XMRV. Patients with a diagnosis of Chronic lyme disease generally fit all of the criteria of Chronic fatigue syndrome.

Results showed that XMRV was detected in 20/20 cases tested.

What is claimed is:

1. A method of detecting, diagnosing, monitoring or managing an Xenotropic Murine Leukemia Virus-Related Virus (XMRV)-related neuroimmune disease or an XMRV-related lymphoma in a subject, comprising:
contacting a sample of a subject and at least one nucleobase polymer under conditions sufficient for hybridization to occur between the at least one nucleobase polymer and an XMRV nucleic acid, or complement thereof, if present in the sample; and
detecting presence, absence or quantity of a hybridization complex comprising the nucleobase polymer and an XMRV nucleic acid, or complement thereof;
wherein the at least one nucleobase polymer comprises a sequence that hybridizes to a nucleic acid sequence comprising at least about 10 contiguous nucleotides of an XMRV nucleic acid, or complement thereof.

2. The method of claim 1, wherein:
the subject is a person having, suspected of having, or at risk for developing an XMRV-related neuroimmune disease or an XMRV-related lymphoma; or
the subject exhibits signs and/or symptoms of a neuroimmune disease and/or a lymphoma.

3. The method of claim 1, wherein:
the neuroimmune disease is selected from the group consisting of Chronic Fatigue Syndrome (CFS), fibromyalgia, Multiple Sclerosis (MS), Parkinson's Disease, Amyotrophic Lateral Sclerosis (ALS), Niemann-Pick Type C Disease, autism spectrum disorder (ASD), and chronic lyme disease; or
the lymphoma is selected from the group consisting of a XMRV-related Mantle Cell Lymphoma (MCL) or a Chronic Lymphocytic Leukemia lymphoma (CLL).

4. The method of claim 1, wherein:
the sample is selected from the group consisting of a blood sample, a serum sample, a plasma sample, a cerebrospinal fluid sample, and a solid tissue sample; or
the sample comprises cells selected from the group consisting of fibroblasts, endothelial cells, peripheral blood mononuclear cells, and haematopoietic cells, or a combination thereof.

5. The method of claim 1, wherein the conditions sufficient for hybridization to occur consists of high stringency hybridization conditions.

6. The method of claim 1, wherein the sequence that hybridizes to a nucleic acid sequence comprising at least 10 contiguous nucleotides of an XMRV nucleic acid, or a complement thereof, comprises the complement of a sequence having at least 80% sequence identity with a sequence comprised by an XMRV virus nucleic acid, or complement thereof.

7. The method of claim 1, wherein the sequence that hybridizes to a nucleic acid sequence comprising at least 10 contiguous nucleotides of an XMRV nucleic acid, or a complement thereof, comprises the complement of a sequence having at least 95% sequence identity with a sequence comprised by an XMRV virus nucleic acid, or complement thereof.

8. The method of claim 1, wherein the sequence that hybridizes to a nucleic acid sequence comprising at least 10 contiguous nucleotides of an XMRV nucleic acid, or a complement thereof, comprises the complement of a sequence comprised by an XMRV virus nucleic acid, or complement thereof.

9. The method of claim 1, wherein the nucleobase polymer comprises DNA, RNA, or a nucleic acid analogue.

10. The method of claim 1, wherein:
the nucleobase polymer further comprises a label selected from the group consisting of a radioisotope, a chromogen, a chromophore, a fluorophore, a fluorogen, an enzyme, a quantum dot and a resonance light scattering particle; and
detecting presence, absence or quantity of the hybridization complex comprises detecting presence, absence or quantity of the label.

11. The method of claim 1, wherein the detecting presence, absence or quantity of the hybridization complex comprises a hybridization assay selected from the group consisting of a Southern hybridization assay, a Northern hybridization assay, a dot-blot hybridization assay, a slot-blot hybridization assay, a Polymerase Chain Reaction (PCR) assay and a flow cytometry assay.

12. The method of claim 11 wherein the PCR assay is a quantitative real time polymerase chain reaction assay.

13. The method of claim 11 wherein the PCR assay comprises one or more primers selected from the group consisting of SEQ ID NOS: 5-20.

14. The method of claim 1, comprising selecting or modifying a treatment on the basis of the detection of the presence, absence or quantity of a hybridization complex comprising the nucleobase polymer and the XMRV nucleic acid sequence or the complement thereof.

15. The method of claim 13 wherein if a hybridization complex is detected, the treatment comprises administrating to the subject a therapeutically effective amount of an anti-viral compound.


SEQUENCE LISTING (56 sequences)

SEQ ID NO: 1 - DNA, 8185 bp, Xenotropic murine lukemia virus related virus
gcgccagtcatccgatagactgagtcgcccgggtacccgtgttcccaataaagccttttgctgtttgcatccgaagcgtggcctcgctgttccttgggagggtctcctcagagtgattgactacccagctcgggggtctttcatttgggggctcgtccgggattcggagacccccgcccagggaccaccgacccaccgtcgggaggtaagccggccggcgatcgttttgtctttgtctctgtctttgtgcgtgtgtgtgtgtgccggcatctaatcctcgcgcctgcgtctgaatctgtactagttagctaactagatctgtatctggcggttccgcggaagaactgacgagttcgtattcccggccgcagccctgggagacgtcccagcggcctcgggggcccgttttgtggcccattctgtatcagttaacctacccgagtcggactttttggagtggctttgttgggggacgagagacagagacacttcccgcccccgtctgaatttttgctttcggttttacgccgaaaccgcgccgcgcgtctgatttgttttgttgttcttctgttcttcgttagttttcttctgtctttaagtgttctcgagatcatgggacagaccgtaactacccctctgagtctaaccttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccctctataaagtccaaacctcctaagccccaggttctccctgatagcggcggacctctcattgaccttctcacagaggatcccccgccgtacggagcacaaccttcctcctctgccagggagaacaatgaagaagaggcggccaccacctccgaggtttcccccccttctcccatggtgtctcgactgcggggaaggagagaccctcccgcagcggactccaccacctcccaggcattcccactccgcatggggggagatggccagcttcagtactggccgttttcctcctctgatttatataattggaaaaataataacccttccttttctgaagatccaggtaaattgacggccttgattgagtccgtcctcatcacccaccagcccacctgggacgactgtcagcagttgttggggaccctgctgaccggagaagaaaagcagcgggtgctcctagaggctagaaaggcagtccggggcaatgatggacgccccactcagttgcctaatgaagtcaatgctgcttttccccttgagcgccccgattgggattacaccactacagaaggtaggaaccacctagtcctctaccgccagttgctcttagcgggtctccaaaacgcgggcaggagccccaccaatttggccaaggtaaaagggataacccagggacctaatgagtctccctcagcctttttagagagactcaaggaggcctatcgcaggtacactccttatgaccctgaggacccagggcaagaaaccaatgtgtccatgtcattcatctggcagtctgccccggatatcggacgaaagttagagcggttagaagatttaaagagcaagaccttaggagacttagtgagggaagctgaaaagatctttaataagcgagaaaccccggaagaaagagaggaacgtatcaggagagaaatagaggaaaaagaagaacgccgtagggcagaggatgagcagagagagagagaaagggaccgcagaagacatagagagatgagcaagctcttggccactgtagttattggtcagagacaggatagacaggggggagagcggaggaggccccaacttgataaggaccaatgcgcctactgcaaagaaaagggacactgggctaaggactgcccaaagaagccacgagggccccgaggaccgaggccccagacctccctcctgaccttaggtgactagggaggtcagggtcaggagcccccccctgaacccaggataaccctcaaagtcggggggcaacccgtcaccttcctggtagatactggggcccaacactccgtgctgacccaaaatcctggacccctaagtgacaagtctgcctgggtccaaggggctactggaggaaagcggtatcgctggaccacggatcgcaaagtacatctggctaccggtaaggtcacccactctttcctccatgtaccagactgcccctatcctctgctaggaagagacttgctgactaaactaaaagcccaaatccactttgagggatcaggagctcaggttgtgggaccgatgggacagcccctgcaagtgctgacagtaaacatagaagatgagtattggctacatgataccaggaaagagccagatgttcctctagggtccacatggctttctgatttccttcaggcctgggcggaaaccgggggcatgggactggcagttcgccaagctcctctgatcatacctctgaaggcaacctctacccccgtgtccataaaacaataccccatgtcacaagaagccagactggggatcaagccccacatacagaggctgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaactgacctggaccagactcccacagggtttcaaaaacagtcccaccctgtttgatgaggcactgcacagagacctagcagatttccggatccagcacccagacttgatcctgctacagtacgtggatgacttactgctggccgccacttctgagcaagactgccaacgaggtactcgggccctattacaaaccctagggaacctcgggtatcgggcctcggccaagaaagcccaaatttgccagaaacaggtcaagtatctggggtatctcctaaaagagggacagagatggctgactgaggccagaaaagagactgtgatggggcagcccactccgaagacccctcgacaactaagggagttcctagggacggcaggcttctgtcgcctctggatccctgggtttgcagaaatggcagcccccttgtaccctcttaccaaaacggggactctgtttaattggggcccagaccagcaaaaggcctatcaagaaatcaaacaggctcttctaactgcccccgccctgggattgccagatttgactaagccctttgaactctttgtcgacgagaagcagggctacgccaaaggcgtcctaacgcaaaaactgggaccttggcgtcggcctgtggcctacctgtccaaaaagctagacccagtggcagctgggtggcccccttgcctacggatggtagcagccattgccgttctgacaaaaaatgcaggcaagctaactatgggacagccgctagtcattctggccccccatgcggtagaagcactggtcaaacaaccccctgaccgttggctatccaatgcccgcatgacccactatcaggcaatgctcctggatacagaccgggttcagttcggaccggtggtggccctcaacccggccaccctgctccccctaccggaaaaggaagccccccatgactgcctcgagatcttggctgagacgcacggaaccagaccggacctcacggaccagcccatcccagacgctgattacacttggtacacagatggaagcagcttcctacaagaaggacaacggagagctggagcagcggtgactactgagaccgaggtaatctgggcgagggctctgccggctggaacatccgcccaacgagccgaactgatagcactcacccaagccttaaagatggcagaaggtaagaagctaaatgtttacactgatagccgctatgccttcgccacggcccatgtccatggagaaatatataggaggcgagggttgctgacctcagaaggcagagaaattaaaaacaagaacgagatcttggccttgctaaaagctctctttctgcccaaacgacttagtataattcactgtccaggacatcaaaaaggaaacagtgctgaggccagaggcaaccgtatggcagatcaagcagcccgagaggcagccatgaaggcagttctagaaacctctacactcctcatagaggactcaaccccgtatacgcctccccatttccattacaccgaaacagatctcaaaagactacgggaactgggagccacctacaatcagacaaaaggatattgggtcctacaaggcaaacctgtgatgcccgatcagtccgtgtttgaactgttagactccctacacagactcacccatctgagccctcaaaagatgaaggcactcctcgacagagaagaaagcccctactacatgttaaaccgggacagaactatccagtatgtgactgagacctgcaccgcctgtgcccaagtaaatgccagcaaagccaaaattggggcaggggtgcgagtacgcggacatcggccaggcacccattgggaagttgatttcacggaagtaaagccaggactgtatgggtacaagtacctcctagtgtttgtagacaccttctctggctgggtagaggcattcccgaccaagcgggaaactgccaaggtcgtgtccaaaaagctgttagaagacatttttccgagatttggaatgccgcaggtattgggatctgataacgggcctgccttcgcctcccaggtaagtcagtcagtggccgatttactggggatcgattggaagttacattgtgcttatagaccccagagttcaggacaggtagaaagaatgaatagaacaattaaggagactttgaccaaattaacgcttgcatctggcactagagactgggtactcctactccccttagccctctaccgagcccggaatactccgggcccccacggactgactccgtatgaaattctgtatggggcacccccgccccttgtcaattttcatgatcctgaaatgtcaaagttaactaatagtccctctctccaagctcacttacaggccctccaagcagtacaacaagaggtctggaagccgctggccgctgcttatcaggaccagctagatcagccagtgataccacaccccttccgtgtcggtgacgccgtgtgggtacgccggcaccagactaagaacttagaacctcgctggaaaggaccctacaccgtcctgctgacaacccccaccgctctcaaagtagacggcatctctgcgtggatacacgccgctcacgtaaaggcggcgacaactcctccggccggaacagcatggaaagtccagcgttctcaaaaccccttaaagataagattaacccgtggggccccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacaggcatactggaagccatcatcatcatgggacctaatttcccttaagcgaggaaacactcctaagggtcagggcccctgttttgattcctcagtgggctccggtagcatccagggtgccacaccggggggtcgatgcaaccccctagtcctagaattcactgacgcgggtaaaagggccagctgggatgcccccaaaacatggggactaagactgtatcgatccactggggccgacccggtgaccctgttctctctgacccgccaggtcctcaatgtagggccccgcgtccccattgggcctaatcccgtgatcactgaacagctacccccctcccaacccgtgcagatcatgctccccaggactcctcgtcctcctccttcaggcgcggcctctatggtgcctggggctcccccgccttctcaacaacctgggacgggagacaggctgctaaacctggtagaaggagcctacctagccctcaacctcaccagtcccgacaaaacccaagagtgctggctgtgtctagtatcgggacccccctactacgaaggggtggccgtcctaggtacttactccaaccatacctctgccccggctaactgctccgtgacctcccaacacaagctgaccctgtccgaagtgaccgggcagggactctgcataggagcagttcccaaaacccatcaggccctgtgtaataccacccagaagacgagcgacgggtcctactatttggcctctcccgccgggaccatttgggcttgcagcaccgggctcactccctgtctatctactactgtgcttaacttaaccactgattactgtgtcctggttgaactctggccaaaggtaacctaccactcccctaattatgtttatggccagtttgaaaagaaaactaaatataaaagagagccggtgtcattaactctggccctgctgttgggaggacttactatgggcggcatagctgcaggagttggaacagggactacagccctagtggccaccaaacaattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgaccctgatatccaccattatgggccctctgatagtacttttattaatcctactcttcggaccctgtattctcaaccgcttggtccagtttgtaaaagacagaatttcggtagtgcaggccctggttctgacccaacagtatcaccaactcaaatcaatagatccagaagaagtggaatcacgtgaataaaagattttattcagtttccagaaagaggggggaatgaaagaccccaccataaggcttagcacgctagctacagtaacgccattttgcaaggcatggaaaagtaccagagctgagttctcaaaagttacaaggaagtttaattaaagaataaggctgaataacactgggacaggggccaaacaggatatctgtagtcaggcacctgggccccggctcagggccaagaacagatggtcctcagataaagcgaaactaacaacagtttctggaaagtcccacctcagtttcaagttccccaaaagaccgggaaataccccaagccttatttaaactaaccaatcagctcgcttctcgcttctgtacccgcgctttttgctccccagtcctagccctataaaaaaggggtaagaactccacactcggcgcgccagtcatccgatagactgagtcgcccgggtacccgtgttcccaataaagccttttgctgtttgcaa

SEQ ID NO: 2 - protein, 1733 aa, Xenotropic murine lukemia virus related virus
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTVNIEDEYWLHDTRKEPDVPLGSTWLSDFLQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKNAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHLSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVSKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 3 - protein, 536 aa, Xenotropic murine lukemia virus related virus
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGD

SEQ ID NO: 4 - protein, 645 aa, Xenotropic murine lukemia virus related virus
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRTPRPPPSGAASMVPGAPPPSQQPGTGDRLLNLVEGAYLALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFTTLISTIMGPLIVLLLILLFGPCILNRLVQFVKDRISVVQALVLTQQYHQLKSIDPEEVESRE

SEQ ID NO: 5 - DNA, 25 bp, Artificial Sequence
  419F PCR primer
atcagttaacctacccgagtcggac

SEQ ID NO: 6 - DNA, 23 bp, Artificial Sequence
  1154R PCR primer
gccgcctcttcttcattgttctc

SEQ ID NO: 7 - DNA, 22 bp, Artificial Sequence
  5922F PCR primer
gctaatgctacctccctcctgg

SEQ ID NO: 8 - DNA, 26 bp, Artificial Sequence
  6273R PCR primer
ggagcccactgaggaatcaaaacagg

SEQ ID NO: 9 - DNA, 25 bp, Artificial Sequence
  4F PCR primer
ccagtcatccgatagactgagtcgc

SEQ ID NO: 10 - DNA, 26 bp, Artificial Sequence
  770R PCR primer
taccatcctgaggccatcctacattg

SEQ ID NO: 11 - DNA, 23 bp, Artificial Sequence
  350F PCR primer
gagttcgtattcccggccgcagc

SEQ ID NO: 12 - DNA, 21 bp, Artificial Sequence
  5135R PCR primer
cctgcggcattccaaatctcg

SEQ ID NO: 13 - DNA, 25 bp, Artificial Sequence
  4789R PCR primer
gggtgagtctgtgtagggagtctaa

SEQ ID NO: 14 - DNA, 25 bp, Artificial Sequence
  4166F PCR primer
caagaaggacaacggagagctggag

SEQ ID NO: 15 - DNA, 24 bp, Artificial Sequence
  7622R PCR primer
ggcctgcactaccgaaattctgtc

SEQ ID NO: 16 - DNA, 27 bp, Artificial Sequence
  4672F PCR primer
gagccacctacaatcagacaaaaggat

SEQ ID NO: 17 - DNA, 25 bp, Artificial Sequence
  7590R PCR primer
ctggaccaagcggttgagaatacag

SEQ ID NO: 18 - DNA, 21 bp, Artificial Sequence
  7472F PCR primer
tcaggacaagggtggtttgag

SEQ ID NO: 19 - DNA, 23 bp, Artificial Sequence
  8182R PCR primer
caaacagcaaaaggctttattgg

SEQ ID NO: 20 - DNA, 19 bp, Artificial Sequence
  8147R PCR primer
ccgggcgactcagtctatc

SEQ ID NO: 21 - DNA, 21 bp, Artificial Sequence
  MCox2-F2 PCR primer
ttctaccagctgtaatcctta

SEQ ID NO: 22 - DNA, 22 bp, Artificial Sequence
  MCox2-R1 PCR primer
gttttaggtcgtttgttgggat

SEQ ID NO: 23 - DNA, 31 bp, Artificial Sequence
  MCox2-PR1 PCR primer
cgtagcttcagtatcattggtgccctatggt

SEQ ID NO: 24 - DNA, 26 bp, Artificial Sequence
  MCox2-P1 PCR primer
ttgctctcccctctctacgcattcta

SEQ ID NO: 25 - DNA, 377 bp, Xenotropic murine lukemia virus related virus
  misc_feature (3)..(3): n is a, c, g, or t
ccngattaccttgcagcactggggagatgtccagcgcattgcatccaaccagtctgtggatgtcaggaagaggcgctggattaccttctgttccgctgaatggccaactttcaatgtgggatggcctcaggatggtactttcaatttaagtattatctctcaggttaagtctagagtgttttgtcctggtccccacggacacccggatcaggtcccatatatcgtcacctgggaggcacttgcctatgacccccccgtgggtcaaaccgtttgtgtctcctaaacttcctcccttgccgacagctcccgtcctcccgcccggtccttctgcgcaacctccgtcccgatctgccctttaccctgcccttaccctctaa

SEQ ID NO: 26 - DNA, 384 bp, Xenotropic murine lukemia virus related virus
  misc_feature (4)..(4): n is a, c, g, or t
ctcngatctaccttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgcccaatacactgcccttacaaaaataaaa

SEQ ID NO: 27 - DNA, 366 bp, Xenotropic murine lukemia virus related virus
ttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccc

SEQ ID NO: 28 - DNA, 366 bp, Xenotropic murine lukemia virus related virus
ttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccctc

SEQ ID NO: 29 - DNA, 366 bp, Xenotropic murine lukemia virus related virus
ttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtattatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccc

SEQ ID NO: 30 - DNA, 366 bp, Xenotropic murine lukemia virus related virus
  misc_feature (364)..(365): n is a, c, g, or t
ttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccnna

SEQ ID NO: 31 - DNA, 366 bp, Xenotropic murine lukemia virus related virus; identical to SEQ ID NO: 28

SEQ ID NO: 32 - DNA, 363 bp, Xenotropic murine lukemia virus related virus
ttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgcccaatacactgcccttaca

SEQ ID NO: 33 - protein, 645 aa, Xenotropic MuLV-related Virus VP35
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRPPRPPPSGAASMVPGAPPPSQQPGTGDRLLNLVEGAYQALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFGKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKKECCFYADHTGVVRDSMAKLRERLNQRQKLFESGQGWFEGLFNRSPWFTTLISTIMGPLIVLLLILLFGPCILNRLVQFVKDRISVVQALVLTQQYHQLKSIDPEEVESRE

SEQ ID NO: 34 - protein, 1733 aa, Xenotropic MuLV-related Virus VP35
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTLSIKSKPPKPQVLPDSGGPLIDLLTEDPPPYGVQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEAGKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIENKYRLHETSKEPDVPLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHPSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVTKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 35 - protein, 536 aa, Xenotropic MuLV-related Virus VP35
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTLSIKSKPPKPQVLPDSGGPLIDLLTEDPPPYGVQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEAGKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGD

SEQ ID NO: 36 - protein, 645 aa, Xenotropic MuLV-related Virus VP42
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRPPRPPPSGAASMVPGAPPPSQQPGTGDRLLNLVEGAYQALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFTTLISTIMGPLIVLLLILLFGPCILNRLVQFVKDRISVVQALVLTQQYHQLKSIDPEEVESRE

SEQ ID NO: 37 - protein, 1733 aa, Xenotropic MuLV-related Virus VP42
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGIISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWGYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVPLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGGSFLQEGQRRAGAAVTTETEVIWGGVLPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHLSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVSKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 38 - protein, 536 aa, Xenotropic MuLV-related Virus VP42
MGQTVTTPLSLTLQHWGDVQRIASNQSVDVKKRRWVTFCSAEWPTFNVGWPQDGTFNLGIISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWGYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGD

SEQ ID NO: 39 - protein, 645 aa, Xenotropic MuLV-related Virus VP62; identical to SEQ ID NO: 4

SEQ ID NO: 40 - protein, 1733 aa, Xenotropic MuLV-related Virus VP62; identical to SEQ ID NO: 2
  misc_feature (537)..(537): Xaa can be any naturally occurring amino acid

SEQ ID NO: 41 - protein, 536 aa, Xenotropic MuLV-related Virus VP62; identical to SEQ ID NO: 3

SEQ ID NO: 42 - protein, 645 aa, Xenotropic MuLV-related Virus VP62
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRPPRPPPSGAASMVPGAPPPSQQPGTGDRLLNLVEGAYQALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESRQGWFEGLFNRSPWFTTLISTIMGPLIVLLLILLFGPCILNRLVQFVKDRISVVQALVLTQQYHQLKSIDPEEVESRE

SEQ ID NO: 43 - protein, 1733 aa, Xenotropic MuLV-related Virus VP62
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVPLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHLSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVSKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 44 - protein, 536 aa, Xenotropic MuLV-related Virus VP62; identical to SEQ ID NO: 3

SEQ ID NO: 45 - protein, 645 aa, Xenotropic MuLV-related Virus VP62; identical to SEQ ID NO: 4

SEQ ID NO: 46 - protein, 1733 aa, Xenotropic MuLV-related Virus VP62; identical to SEQ ID NO: 2
  misc_feature (537)..(537): Xaa can be any naturally occurring amino acid

SEQ ID NO: 47 - protein, 536 aa, Xenotropic MuLV-related Virus VP62; identical to SEQ ID NO: 3

SEQ ID NO: 48 - protein, 409 aa, Friend Spleen Focus-Forming Virus (isolate 502)
MKGPAFSKPLKDKINPWGPLIVLGILIRAGVSVQHDSPHQVFNVTWRVTNLMTGQTANATSLLGTMTDAFPMLHFDLCDLIGDDWDETGLECRTPGGRKRARTFDFYVCPGHTVPTGCGGPREGYCGKWGCETTGQAYWKPSSSWDLISLKRGNTPKDRGPCYDSSVSSGVQGATPGGRCNPLVLKFTDAGKKASWDSPKVWGLRLYRPTGIDPVTRFSLTRQVLNIGPRIPIGPNPVIIGQLPPSRPVQVRLPRPPQPPPTGAASMVPGTAPPSQQPGTGDRLLNLVQGAYQALNLTNPDKTQECWLCLVSGPPYYEGVAVLGTNSNHTSALKEKCCFYADHTGLVRDSMAKLRKRLTQRQKLFESSQGWFEGSFNRSPWFTTLISTIMGLLIILLLLLILLLWTLHS

SEQ ID NO: 49 - protein, 187 aa, Friend Spleen Focus-Forming Virus (isolate 502)
MGQTVTTPLSLTLEHWEDVQRTASNQSVDVKKRRWVTFCSAEWPTFGVGWPQDGTFNLDIILQVKSKVFSPGPHGHPDQVPYIVTWEAIAYEPPPWVKPFVSPKLSPSPTAPILPSGPSTQPPPRSALYPALTPSIKPGPSPIMADLSLTFSQKTLRRTEDRDRPPLTEMATEKRPPPLLRFLPPLP

SEQ ID NO: 50 - protein, 356 aa, Friend Spleen Focus-Forming Virus (strain BB6)
MEGPAFSKPLKDKINPWGPLIVLGILIRAGVSVQRDSPHQVFNVTWRVTNLMTGQTANATSLLGTMTDAFPKLYFDLCDLIGNDWDETRLGCRTPGEGKRARTFDLYVCPGHTVPTGCGGPREGYCGKWGCETTGQAYWKPSSSWDLISLKRGNTPKDRGPCYDSSVSSGVQGATPGGRCNPLVLKFTDAGKKASWDAPKVWGLRLYRSTGTDPVTRFSLTRQVLNIGPRVPIGPNPVISDQLPPSRPAQIMLPRPPQPPPPGTASIVPETAPPSQQPGTRDRLLNLVNKAYQALNLTSPDKTQECWLCLVSRPPYYEGVAVLGTNSNHTTLISTIMGLLIILLLLLILLLWTLHS

SEQ ID NO: 51 - protein, 409 aa, Friend Spleen Focus-Forming Virus (strain Lilly-Steeves)
MEGPASSKPLKDKTNPWGPLIILGILIRAGVSVQLDSPHQVSNVTWRVTNLMTGQTANATSLLGTMTEAFPKLYFDLCDLMGDDWDETGLGCRTPGGRKRARTFDFYVCPGHTVPTGCGGPREGYCGKWGCETTGQAYWKPSSSWDLISLKRGNTPKDQGPCYDSSVSSGVLGATPGGRCNPLVLEFTDAGRKASWDAPKVWGLRLYRSTGTDPVTRFSLTRQVLDIGPRVPIGSNPVTTDQLPLSRPVQTMPPRPLQPPPPGAASIVPETAPPPQQPGAGDRLLNLVDGAYQALNLTNPDKIQECWLCLVSGPPYYEGVVVLGTYFNHTIALKEKCCFYADHTGLVRDSMAKLRKRLTQRQKLFESSRGWFEGSSNRSPWFTTLISAIMGSLIILLLLLILLIWTLYS

SEQ ID NO: 52 - protein, 408 aa, Rauscher Spleen Focus-Forming Virus
MEGPAFSKPLKDKINPWGPLIILGILIRAGVSVQHDSPHQVFNVTWRVTNLMTGQTANATSLLGTMTDAFPKLYFDLCDLIGDDWDETGLGCRTPGGRKRARTFDFYVCPGHTVPTGCGGPREGYCGKWGCETTGQAYWKPSSSWDLISLKRGNTPRNQGPCYDSSAVSSDIKGATPGGRCNPLVLEFTDAGKKASWDGPKVWGLRLYRSTGTDPVTRFSLTRQVLNIGPRVPIGPNPVITDQLPPSRPVQIMLPRPPQPPPPGAASIVPETAPPSQQPGTGDRLLNLVDGAYQALNLTNPDKTQDCWLCLVSGPPYYEGVAVLGTYYNHTSALKEECCFYADHTGLVRDSMAKLRERLTQRQKLFESSQGWFEELFNRSTWFTTLIFTIIGPLIILLLILLFWTLHS

SEQ ID NO: 53 - protein, 116 aa, Rauscher Spleen Focus-Forming Virus
AHLHALYLVHHEVWRPLAAAYQHQLDRPIVPHPFRLGDTVWVRRHQTNNLQPRWKAPYTVLLTTPTALKVDGIAAWIHAAHVKAATTPPAGTASGPTWKVQRSQNPLKIRLTRGAP

SEQ ID NO: 54 - protein, 27 aa, Rauscher Spleen Focus-Forming Virus
YNHTSALKRECCFYADHTGLVRDSMAK

SEQ ID NO: 55 - protein, 408 aa, Rauscher Spleen Focus-Forming Virus; identical to SEQ ID NO: 52

SEQ ID NO: 56 - protein, 116 aa, Rauscher Spleen Focus-Forming Virus; identical to SEQ ID NO: 53